(19) United States
(12) Patent Application Publication
(10) Pub. No.: US 2005/0058622 A1
(43) Pub. Date: Mar. 17, 2005

(54) Methods of using Flt3-Ligand in hematopoietic cell transplantation procedures incorporating nonmyeloablative conditioning regimens

(76) Inventors: Stewart D. Lyman, Seattle, WA (US); M. Patricia Beckmann, Hansville, WA (US); Hilary J. McKenna, Seattle, WA (US); Richard A. Nash, Seattle, WA (US)

Correspondence Address: Immunex Corporation;Law Department, 1201 Amgen Court West, Seattle, WA, 98119, US

(21) Appl. No.: 10/730,334

(22) Filed: Dec. 8, 2003

Related U.S. Application Data
(60) Provisional application No. 60/431,266, filed on Dec. 6, 2002.

(51) Int. Cl. A61K 38/17; A61K 45/00

(52) U.S. Cl. 424/85.1; 514/12; 424/93.7

(57) ABSTRACT

The invention is directed to methods of using Flt3-Ligand in hematopoietic cell transplantation procedures using nonmyeloablative conditioning regimens. This abstract is provided for the sole purpose of enabling the reader to quickly ascertain the subject matter of the technical disclosure and is not intended to be used to interpret or limit the scope or meaning of the claims.

### CROSS REFERENCE TO RELATED APPLICATIONS

This application claims the benefit under 35 U.S.C. §119 of U.S. Provisional Application Ser. No. 60/431,266, filed Dec. 6, 2002.

### FIELD OF THE INVENTION

The present invention relates to the use of Flt3-L in hematopoietic cell transplantation procedures incorporating nonmyeloablative conditioning regimens for the treatment of various malignancies.

### BACKGROUND OF THE INVENTION

Cancer is typically treated with cytoreductive therapies that involve administration of ionizing radiation or chemical toxins that kill rapidly dividing cells. Side effects typically result from cytotoxic effects upon normal cells and can limit the use of cytoreductive therapies. A frequent side effect is myelosuppression, or damage to bone marrow cells that give rise to white and red blood cells and platelets. As a result of myelosuppression, patients develop cytopenia, or blood cell deficits, that increase risk of infection and bleeding disorders.

Cytopenias increase morbidity, mortality, and lead to under-dosing in cancer treatment. Many clinical investigators have manipulated cytoreductive therapy dosing regimens and schedules to increase dosing for cancer therapy, while limiting damage to bone marrow. One approach involves bone marrow or peripheral blood cell transplants in which bone marrow or circulating hematopoietic progenitor or stem cells are removed before cytoreductive therapy and then reinfused following therapy to restore hematopoietic function. U.S. Pat. No. 5,199,942, incorporated herein by reference, describes a method for using GM-CSF, IL-3, SF, GM-CSF/IL-3 fusion proteins, erythropoietin (“EPO”) and combinations thereof in autologous transplantation regimens.

Myelodysplastic syndromes are stem cell disorders characterized by impaired cellular maturation, progressive pancytopenia, and functional abnormalities of mature cells. They have also been characterized by variable degrees of cytopenia, ineffective erythropoiesis and myelopoiesis with bone marrow cells that are normal or increased in number and that have peculiar morphology. Treatment of theses syndromes with retinoids, vitamin D, and cytarabine has not been successful. Most of the patients suffering from these syndromes are elderly and are not suitable candidates for bone marrow transplantation or aggressive antileukemic chemotherapy.

Aplastic anemia is another disease entity that is characterized by bone marrow failure and severe pancytopenia. Unlike myelodysplastic syndrome, the bone marrow is acellular or hypocellular in this disorder. Current treatments include bone marrow transplantation from a histocompatible donor or immunosuppressive treatment with antithymocyte globulin (ATG). Similarly to myelodysplastic syndrome, most patients suffering from this syndrome are elderly and are unsuitable for bone marrow transplantation or for aggressive antileukemic chemotherapy. Mortality in these patients is exceedingly high from infectious or hemorrhagic complications.

Allogeneic hematopoietic stem cell transplantation (aHSCT) is an established treatment for patients with hematologic malignancies, but with strict limitations in its application. Historically, aHSCT employed maximally tolerated doses of systemic chemotherapy and/or radiotherapy to eradicate the malignancy and allografts to rescue the patient's hematopoietic system from treatment-induced aplasia. Unfortunately, the toxicities associated with this therapy have severely limited aHSCT treatment to younger, medically fit patients. Almost no aHSCTs have been done in patients more than 60 years of age, and relatively few in patients older than 50 years (Molina, et al., Handbook of Bone Marrow Transplantation, London, UK; Martin Dunitz Ltd., 2000:111-137). Consequently, median patient ages at aHSCT for chronic myelocytic leukemia (CML), chronic lymphocytic leukemia (CLL), acute myelocytic leukemia (AML), multiple myeloma (MM), and non-Hodgkin lymphoma (NHL) are approximately 2 decades younger than median ages at diagnoses. Thus, current aHSCT benefits only a younger minority of patients with candidate diseases (MacSweeney, et al., Blood, vol. 97, no. 11, 2001:3390-3400).

Studies have shown that cures may be partially attributed to graft-versus-tumor reactions mediated by donor T cells (Horowitz, et al., Blood, vol. 75, no. 3, 1990: 555-562). In addition, donor lymphocyte infusions can induce sustained complete remissions of malignancies that relapsed after aHSCT (Kolb, et al., Blood, vol. 86, no. 5, 1995: 2041-2050). More recently, the emphasis in treating hematopoietic malignancies is shifting away from relying on high-dose cytotoxic therapy.

The present invention builds upon these advances by providing unique hematopoietic cell transplantation therapies for treating hematopoietic malignancies that are more effective and less detrimental than conventional therapies.

### SUMMARY OF THE INVENTION

Embodiments of the invention include methods of using Flt3-Ligand (Flt3-L) in hematopoietic cell transplantation procedures, wherein the hematopoietic cells are derived from bone marrow and/or peripheral blood. The hematopoietic cells derived from bone marrow and/or peripheral blood may be from autologous bone marrow and/or autologous peripheral blood, allogeneic bone marrow and/or allogeneic peripheral blood or syngeneic bone marrow and/or syngeneic peripheral blood. Further embodiments include methods of using Ft3-L in hematopoietic transplantation procedures wherein the hematopoietic stem and/or progenitor cells are obtained from umbilical cord blood. The hematopoeitic cells may comprise hematopoietic stem cells, hematopoietic progenitor cells, as well as combinations of hematopoietic stem and progenitor cells, as well as intermediate cell types.

Further embodiments include methods of treating a patient in need of a hematopoietic cell transplant comprising administering a Flt3-L composition to the patient, subjecting the patient to cytoreductive therapy and transplanting hematopoietic cells to the patient. The Flt3-L may be administered prior to, concurrent with and/or subsequent to transplanting hematopoietic cells.

Additional embodiments are directed to methods treating a patient in need of a hematopoietic cell transplant by administering Flt3-L in hematopoietic cell transplantation methods that include cytoreductive therapy that is nonmyeloablative. For example, Flt3-L may be used in a method to treat a patient having a hematopoietic malignancy comprising administering a Flt3-L composition to the patient, administering a nonmyeloablative conditioning regimen and transplanting hematopoietic cells to the patient.

Additional embodiments are directed to methods of using Flt3-L in hematopoietic cell transplantation methods that include some form of a nonmyeloablative conditioning regimen and the patient is administered an immunosuppressive agent pre- or post-transplant.

Additional embodiments are directed to methods of using Flt3-L in hematopoietic cell transplantation methods that include some form of a nonmyeloablative conditioning regimen and the patient is administered some form of adoptive immunotherapy post-transplant, such as Donor Lymphocyte Infusions. Of course, the patient may also be treated with one or more immunosuppressive agents post-transplant in addition to the adoptive immunotherapy.

In a further embodiment, the bone marrow or peripheral blood stem and/or progenitor cells are expanded in an ex vivo procedure prior to administration to the patient. In a particular embodiment, autologous hematopoietic cells are removed from a patient prior to a nonmyeloablative conditioning regimen, and then re-administered to the patient concurrent with or following cytoreductive therapy to counteract the myelosuppressive effects of such therapy. The present invention provides for the use of Flt3-L in at least one of the following manners: (i) Flt3-L is administered to the patient prior to collection of the progenitor and/or stem cells to increase or mobilize the numbers of such circulating cells; (ii) following collection of the patient's progenitor or stem cells, Flt3-L is used to expand such cells ex vivo; and optionally (iii) Flt3-L is administered to the patient following transplantation of the collected progenitor or stem cells to facilitate engraftment thereof. The transplantation method may further comprise the use of an effective amount of a cytokine/growth factor in sequential or concurrent combination with the Flt3-L. Such cytokines/growth factors include, but are not limited to interleukins (“IL”) IL-1, IL-2, IL-3, IL-4, IL-5, IL-6, IL-7, IL-8, IL-9, IL-10, IL-11, IL-12, IL-13, IL-14 or IL-15, a CSF selected from the group consisting of G-CSF, GM-CSF, M-CSF, or GM-CSF/IL-3 fusions, or other growth factors such as CSF-1, SF, EPO, leukemia inhibitory factor (“LIF”) or fibroblast growth factor (“FGF”). The aforementioned methods are also useful for syngeneic or allogeneic transplantations.

The present invention also pertains to the use of Flt3-L agonists, such as agonistic antibodies, that mimic the effect of Flt3-L polypeptides binding to the Flt3 receptor. In particular monoclonal antibodies, that are immunoreactive with Flt3 and especially human or fully humanized monoclonal antibodies. Fusion proteins comprising a soluble portion of Flt3-L and the constant domain of an immunoglobulin protein are also embodied in the invention. These Flt3-L agonists, chimeric or fusion proteins as well as Flt3-L derivatives may be used in all of the methods described herein.

Pharmaceutical compositions may comprise Flt3-L alone or in combination with other cytokines and/or growth factors, such as interleukins, colony stimulating factors, protein tyrosine kinases and cytokines. For example, an interleukin family member (e.g., IL-1, IL-2, IL-3, IL-4, IL-5, IL-6, IL-7, IL-8, IL-9, IL-10, IL-11, IL-12, IL-13, IL-14 or IL-15, IL-16, IL-17, IL-18, IL-19, IL-20, IL-21, IL-22, IL-23, IL-24, IL-25) an interferon family member (alpha, beta or gamma), a CSF selected from the group consisting of G-CSF, GM-CSF, M-CSF, or GM-CSF/IL-3 fusions, or other growth factors such as SCF, EPO, leukemia inhibitory factor (“LIF”) or fibroblast growth factor (“FGF”).

Further embodiments include methods of using Flt3-L in autologous, allogeneic or syngeneic hematopoietic cell transplantation methods incorporating a nonmyeloablative conditioning regimen, comprising administering a Flt3-L composition to a patient in need of a hematopoietic cell transplant and transplanting hematopoietic cells to the patient, wherein the method further comprises the step of administering one or more additional cytokines/growth factors, wherein the growth factor is selected from the group consisting of an interleukin family member (e.g., IL-1, IL-2, IL-3, IL-4, IL-5, IL-6, IL-7, IL-8, IL-9, IL-10, IL-11, IL-12, IL-13, IL-14 or IL-15, IL-16, IL-17, IL-18, IL-19, IL-20, IL-21, IL-22, IL-23, IL-24, IL-25) an interferon family member (alpha, beta or gamma), a CSF selected from the group consisting of G-CSF, GM-CSF, M-CSF, or GM-CSF/IL-3 fusions, or other growth factors such as SCF, EPO, leukemia inhibitory factor (“LIF”) or fibroblast growth factor (“FGF”). The Flt3-L and one or more of the cytokones/growth factors listed above may be administered prior to, concurrent with and/or subsequent to transplanting hematopoietic cells.

Additional embodiments are directed to methods treating a autoimmune disease by administering Flt3-L in the context of an hematopoietic cell transplantation that includes a nonmyeloablative conditioing regimen.

Embodiments of the invention also pertain to biologically active Flt3-L polypeptides and pharmaceutical compositions thereof.

The various embodiments of the invention are not limited by the foregoing summary. Additional embodiments are provided in the sections that follow.

### BRIEF DESCRIPTION OF THE DRAWINGS

FIG. 1 is a table summarizing the data for the transplantation of DLA-matched marrow after treatment with Flt3-L and 4.5 Gy total body irradiation (TBI).

FIG. 2 shows that PBMC from Flt3-L-treated dogs increased proliferation of responder cells in MR. Shown are the mean values of ³H-thymidine incorporation with PBMC from three Flt3-L-treated healthy dogs as stimulators. Autologous control PBMC as responders to irradiated stimulator PBMC obtained before (A) and after 13 days of Flt3-L (B). PBMC from three unrelated DLA-mismatched dogs as responders to irradiated stimulator PBMC before (C) and after 13 days of Flt3-L (D). (n=number of experiments conducted)

FIG. 3 shows absolute neutrophil recovery in nine Flt3-L-treated dogs after 4.5 Gy TBI and marrow transplantation from DLA-identical littermates. All dogs had a median neutrophil count of 580 cells/μL (range, 91-1,020 cells/μL) at 7±1 days post-transplant. Dogs E929 and E873 showed secondary nadir after initial recovery. Dog E929 rejected the graft at week 6; dog E873 became low-degree (8% in whole blood) stable mixed chimera.

FIG. 4 illustrates the percentage of donor chimerism in Flt3-L-treated dogs after 4.5 Gy TBI and marrow transplantation from DLA-identical littermates. DNA from unfractionated hemolyzed peripheral blood was obtained at various time points after transplant. Engraftment is stable for a follow-up period up to 72 weeks. Representative example of mixed donor-host hematopoietic chimerism in dog E945 at 36 and 53 weeks after marrow transplant. Skin grafting from marrow donor, unrelated DLA-mismatched dog, and autologous control at week 40 (arrow). Autologous skin graft and skin graft from BM donor were accepted in the recipient, whereas third skin graft from an unrelated dog was acutely rejected. Microsatellite marker analysis of the DNA extracted from the BM donor skin graft biopsy specimen and pulled-out hair confirmed donor origin of the graft.

### DETAILED DESCRIPTION OF THE INVENTION

Allogeneic hematopoietic cell transplantation is used for the treatment of selected hematological malignancies and in some instances is the only effective treatment for hematologic malignancies resistant to conventional chemotherapy. Its curative potential is based on two very different mechanisms, involving the conditioning regimen and the graft-versus-host reactions. In traditional transplant therapies, the high-dose chemo-radiotherapy conditioning regimen was aimed at destroying tumor cells, ablating the host immune system (to prevent rejection) and eliminating the host bone marrow (to “make space” for donor stem cells). Due to its toxicity, conventional allogeneic HSCT is restricted to younger (<55 years) and fitter patients. In addition, it is known in the art that certain malignancies involving the bone marrow are frequently not eradicated by such myeloablative regimens. Importantly, the definitive eradication of tumor cells is largely mediated by a graft-versus-malignancy (GVM) process, whereby allo-reactive donor T-lymphocytes target the destruction of malignant cells.

These observations led researchers to design less toxic transplant protocols based on a two step approach: first the use of nonmyeloablative immunosuppressive conditioning regimens providing sufficient immunosuppression to achieve engraftment of allogeneic hematopoietic stem cells and, in a second step, destruction of malignant cells by the GVM effect. These transplants are called nonmyeloablative HSCT or reduced-conditioning HSCT or minitransplants (Beguin, Y., et al., Acta Clin Belg. 2003; 58(1): 37-45).

Recipient-derived antigen-presenting cells have been shown to be critical for initiating a GVH reaction (Schlomchik, W., et al., Science 1999; 285: 412). Dendritic cells (DC) are the most efficient type of professional antigen-presenting cells. In humans, Flt3-L is a crucial cytokine that induces expansion of both hematopoietic progenitor and stem cells and DC when administered in vivo. Flt3-L is also a crucial cytokine for the ex vivo expansion of DC in dogs and other species. In mice, Flt3-L treatment increases alloimmune reactivity and augments chimerism levels of donor-derived hematopoietic cells after both solid-organ transplantation and HSCT.

Without being bound by theory, it was hypothesized that enhancement of GVH reactions with Flt3-L-augmented host antigen presentation could promote engraftment. This was shown to be true in a canine model of dog leukocyte antigen (DLA)-identical allogeneic bone marrow (BM) transplantation after a reduced dose of total-body irradiation (TBI) (4.5 Gy) without post-transplant immunosuppression. Previously in this model, engraftment had been promoted with the addition of a monoclonal antibody against the T-cell receptor (TCR)-alpha-beta before transplantation or cyclosporine (CSP) after transplantation (Barsoukov, A, et al., Transplantation 1999; 67: 1329 and Yu, C, et al., Blood 1995; 86: 4376-, respectively). Augmentation of the marrow graft with donor peripheral blood mononuclear cells, treatment of the recipient with CSP before transplant, and treatment of the recipient with other hematopoietic growth factors or prednisone after transplantation did not increase the probability of engraftment in this model (Storb, R., et al., Blood 1994; 84: 3558 and Storb, R., et al., Transplantation 1995; 59: 1481, respectively).

Example 6 describes the successful use of Flt3-L in a nonmyeloablative conditioning regimen and allogeneic bone marrow transplantation. In brief, Flt3-L was first administered to three nonirradiated healthy dogs for 13 days at a dosage of 100 μg/kg/day. Next, nine dogs received 4.5 Gy total-body irradiation (TBI) and unmodified marrow grafts from dog leukocyte antigen (DLA)-identical littermates without post-transplant immunosuppression. Flt3-L was administered to the recipients at a dosage of 100 μg/kg/day from day −7 until day +5. The results show that in normal dogs, Flt3-L produced significant increases in monocytes (CD14⁺) and neutrophils in the peripheral blood, a marked increase in CD1c⁺ cells with DC-type morphology in lymph nodes, and increased alloreactivity of third-party responders to peripheral blood mononuclear cells in mixed lymphocyte reactions (P<0.001). Sustained engraftment was observed in eight of nine (89%) Flt3-L-treated dogs compared with 14 of 37 (38%) controls (P=0.02, logistic regression). All engrafted Flt3-L-treated dogs became stable complete (n=2) or mixed (n=6) hematopoietic chimeras without significant graft-versus-host disease (GVHD). Recipient chimeric dogs (n=4) were tolerant to skin transplants from their marrow donors but rejected skin grafts from unrelated dogs within 7 to 9 days (median, 8 days).

These studies show that Flt3-L administered to recipients promotes stable engraftment of allogeneic bone marrow from DLA-identical littermates after nonmyeloablative conditioning without significant GVHD. The methods used herein are unique in that they potentially permit one to further reduce the intensity of pre-transplantation and possibly post-transplantation immunosuppressive and myelosuppressive therapy, thereby reducing toxicities usually associated with allografting. These studies also suggest that the use of Flt3-L in hematopoietic cell transplants using nonmyeloablative conditioning regimens is an effective treatment for hematopoietic malignancies. These and other features of the invention will be elaborated upon in the sections that follow.

I. Therapeutic Applications

A. Methods of Using Flt3-Ligand in Hematopoietic Cell Transplantation Procedures Incorporating Nonmyeloablative Conditioning Regimens

Embodiments include methods of using Flt3-L in autologous, allogeneic or syngeneic hematopoietic cell transplantation methods, comprising administering a Flt3-L composition to a patient in need of a hematopoietic cell transplant, administering some form of a nonmyeloablative conditioning regimen and transplanting hematopoietic cells to the patient. Embodiments include methods of using Flt3-L in autologous, allogeneic or syngeneic hematopoietic cell transplantation methods, comprising administering a Flt3-L composition to a patient in need of a hematopoietic cell transplant, administering some form of a nonmyeloablative conditioning regimen and transplanting hematopoietic cells to the patient and optionally administering some form of immunosuppression post-transplant (such as an immunosuppressive agent). Embodiments also include methods of using Flt3-L in autologous, allogeneic or syngeneic hematopoietic cell transplantation methods, comprising administering a Flt3-L composition to a patient in need of a hematopoietic cell transplant, administering some form of a nonmyeloablative conditioning regimen and transplanting hematopoietic cells to the patient and optionally administering some form of immunosuppression post-transplant and optionally administering some form of adoptive immunotherapy to the patient, such as Donor Lymphocyte Infusions. The methods described herein include administering Flt3-L prior to, concurrent with and/or subsequent to transplanting hematopoietic cells.

The hematopoietic cells used in the transplant may comprise, but are not limited to, hematopoietic stem cells, hematopoietic progenitor cells, as well as combinations of hematopoietic stem and progenitor cells. It is understood that the hematopoeitic cells used in the transplant may comprise other cells besides hematopoietic stem and progenitor cells, such as, but not limited to, any monocytes, macrophages, myeloid-related dendritic cells, neutrophils, platelets, erythrocytes, eosinophils, basophils, mast cells, T-lymphocytes, B-lymphocytes, plasma cells, lymphoid-related dendritic cells and the like. It is also understood that there is no consensus in the art as to the exact distinctions, such as phenotype, between a stem cell and a progenitor cell and the numerous intermediates along the various differentiation pathways of these cells. Therefore, when the term “hematopoietic cells” is used herein, such as in the context of a hematopoietic cell transplantation, it encompasses all the cells that would typically be included in a hematopoietic transplantation procedure (most frequently referred to generically as hematopoietic stem cells). Such information is known in the art and need not be reiterated here.

The term “autologous transplantation” means a method in which hematopoietic cells, such as bone marrow- or peripheral blood-derived stem cells and/or progenitor cells are removed from a subject and readministered to the same subject. The term “allogeneic transplantation” means a method in which hematopoietic cells, such as bone marrow- or peripheral blood-derived stem cells and/or progenitor cells are removed from a subject and administered to a genetically different subject of the same species. The term “syngeneic transplantation” means the hematopoietic cells, such as bone marrow- or peripheral blood-derived stem cells and/or progenitor cells are transplanted between genetically identical subjects.

A patient in need of a hematopoietic cell transplant is to be determined by a qualified physician, but in its most general sense, includes patients having a disease or disorder such as, but not limited to, a hematopoietic or hematological malignancy, myelodysplastic syndrome, myeloproliferative disorders, leukemic disorders, and the like. A more complete list of potential therapeutic areas for the methods described herein is provided in the therapeutic applications section below.

The terms nonmyeloablative, nonmyeloablative conditioning regimen and reduced intensity conditioning pertain to essentially the same procedure and several are known in the art. When these terms are used in this application, they are meant in their broadest sense and refer to cytoreductive therapies that do not induce total myeloablation of the immune and/or hematopoietic systems. For example, a nonmyeloablative conditioning regimen comprises a form of cytoreductive therapy, such as ionizing radiation, chemotherapy and/or immunosuppression that is administered at a dose that is adjusted to the individual such that the recipient does not have suffer pronounced cytopenia and/or prolonged myelosuppression. In a more specific example involving a patient having leukemia, a nonmyeloablative conditioning regimen is tailored to facilitate host-versus-graft tolerance for engraftment of donor hematopoietic cells for induction of graft-versus-leukemia effects to displace residual malignant or genetically abnormal host cells.

It is known in the art that the minimum dose of the most commonly used agents needed to induce immunosuppression and allow engraftment of donor cells has not been well established. It may vary among patients, depending on the degree of HLA compatibility, stem cell dose, T-cell content of the graft, primary diagnosis, and prior therapy. Thus, the development of nonmyeloablative immunosuppressive regimens needs to be individualized, depending on the underlying disorder and donor characteristics, and should consist of a combination of agents that are immunosuppressive and effective against the malignant disease. Only a qualified physician will design a nonmyeloablative immunosuppressive regimen tailored to suit an individual's condition.

As reported by Tabbara, et al., (Exp Hematology 31:7, 2003: 559-566), the degree of immunosuppression required to allow engraftment of donor cells depends on histocompatibility, source of hematopoietic stem cells (related vs unrelated), and the immunologic status of the patient. Many of the reported regimens that are labeled as nonmyeloablative have not been shown to induce a reversible myeloablation. This is demonstrated by the need for a stem cell rescue to reconstitute hematopoiesis and by the development of prolonged marrow aplasia when graft rejection occurred. Based on these observations, such regimens may represent reduced toxicity and intensity ablative regimens. Many of the nonmyeloablative regimens that were reported used purine-analog-based chemotherapy such as fludarabine, cladribine, and pentostatin. Purine analogs were chosen because of their effectiveness against hematologic malignancies, their highly immunosuppressive effect with associated lymphocytopenia, and their mild nonhematologic toxicity. In other forms of nonmyeloablative therapies, the use of low-dose single-fraction TBI (2 Gy) without chemotherapy was successfully used, followed by post-transplant immunosuppression with cyclosporine A (CSA) and mycophenolate mofetil (MMF). The initial study enrolled only patients with an HLA-identical sibling donor; unrelated HLA-matched transplants were included later but with the addition of fludarabine and extended use of postgrafting immunosuppression with CSA and MMF because of the high initial rate of graft rejection (Niederwieser, D., et al., Blood 96 suppl 1 (2000), p. 413a). In most reported studies, blood stem cells were used rather than marrow-derived stem cells in view of the data indicating a more rapid engraftment, reduced rate of early mortality, and presence of a 10-fold higher lymphocyte dose in blood stem cells compared to bone marrow, which potentially may enhance the development of a GVM effect. A short course of immunosuppression was included in most of these regimens, and subsequent DLIs were administered to increase the GVM effect in some of the trials. Embodiments of the present invention would use these and other forms of nonmyeloablative conditioning. In one particular embodiment of the invention, CSA and MMF are used in combination for inducing immunosuppression.

With regards to the radiotherapy that may be used in the nonmyeloablative conditioning regime, it is understood that this may encompass any approved form of radiotherapy prescribed by a qualified physician, such as but no limited to particle beams or electromagnetic waves. This includes X-rays, gamma-rays and neutron beams. The radiotherapy may be localized or total body irradiation (TBI). The dosage is to be determined by the physician, but may include a dose anywhere in the range of 0.1 to 100 Gy. The radiotherapy may be used in combination with radiosensitizers and/or radioprotectors as known in the art. The radiotherapy may also comprise the use of some form of radioimmunotherapy, such as radio-labeled antibodies that target malignant cells.

Several potential advantages are associated with the use of Flt3-L in nonmyeloablative stem cell transplantation. The use of Flt3-L in allogeneic hematopoietic cell transplantation using nonmyeloablative conditioning regimens facilitates the allogeneic graft-vs-malignancy phenomena and instills stable engraftment, as well as inducing mixed chimerism and graft-host tolerance. Due to this effect, there may be an increase in the survival rate in transplant recipients. The methods provided herein would allow a broader patient population to be eligible for hematopoietic cell transplants. This would include older and more debilitated patients, who are considered ineligible for conventional myeloablative therapy, as well as very young patients. The methods provided herein use less chemo- and radiotherapeutics and immunosuppressive agents, and as a result, the patient is less immunosuppressed and better able to resist bacterial, viral and fungal infection during and after treatment. In addition, it has been observed that certain cancer drugs selectively promote specific infections, such as tacrolimus with polyoma virus, mecophenolate mofetil with cytomegalovirus and sirolimus with Pneumocystis carinii. Therefore, using a lower dose of these agents may reduce the likelihood of infection associated with those agents. The incidence and severity of acute GVHD may be reduced because its clinical manifestations may be partly caused by intensive chemoradiotherapy-induced tissue injury and subsequent cytokine release. With the methods described herein, not all host lymphocytes are eliminated, and the residual cells may limit or inhibit the development of acute and chronic GVHD. Neutropenic infections are likely to be reduced significantly in recipients. Furthermore, the GVM effect alone can be curative in certain malignancies, such as CML, chronic lymphocytic leukemia, and low-grade lymphomas, and Flt3-L is predicted to facilitate the GVM effect. An additional advantage of the methods described herein is the possibility of reducing or eliminating the need for post-transplant immunosuppression. A further advantage is the possibility of using HLA-matched unrelated donors rather than HLA-identical/HLA-matched sibling donors, which would greatly expand the donor and recipient pools. Embodiments of the invention include the use of hematopoietic cells isolated from an HLA-matched donor (related or unrelated), HLA-partially matched donor (related or unrelated), HLA-mismatched donor (related or unrelated).

Embodiments of the invention include methods comprising administering a Flt3-L composition to a patient in need of a hematopoietic cell transplant, administering cytoreductive therapy and transplanting hematopoietic cells into the patient, wherein the cytoreductive therapy is a nonmyeloablative. Embodiments of the present invention include methods of using Flt3-L in hematopoietic cell transplantation procedures, wherein the hematopoietic cells are derived from bone marrow and/or peripheral blood. The hematopoietic cells derived from bone marrow and/or peripheral blood may be from autologous bone marrow and/or autologous peripheral blood, allogeneic bone marrow and/or allogeneic peripheral blood or syngeneic bone marrow and/or syngeneic peripheral blood. Further embodiments include methods of using Flt3-L in hematopoietic transplantation procedures wherein the hematopoietic cells are obtained from umbilical cord blood.

Additional embodiments include methods comprising administering a Flt3-L composition to a patient in need of a hematopoietic cell transplant, administering a form of a nonmyeloablative conditioning regimen, transplanting hematopoietic cells into the patient, and administering one or more immunosuppressive agents to the patient post-transplantation. The Flt3-L composition may be administered prior to, concurrent with and/or subsequent to cytoreductive therapy and hematopoietic cell transplant. Examples of immunosuppressants are described below.

Additional embodiments include methods comprising administering a Flt3-L composition to a patient in need of a hematopoietic cell transplant, such as when the patient has a hematopoietic malignancy, administering a form of a nonmyeloablative conditioning regimen, wherein the patient does not receive post-transplant immunosuppression. The Flt3-L composition may be administered prior to, concurrent with and/or subsequent to cytoreductive therapy and hematopoietic cell transplant.

The methods described herein use Flt3-L compositions that are described in detail below and in the patents incorporated by reference. Flt3-ligand compositions, which include Flt3-L pharmaceutical compositions, are described in detail below and may further comprise other cytokines and/or growth factors, such as interleukins, colony stimulating factors, protein tyrosine kinases and cytokines. For example, an interleukin family member (e.g., IL-1, IL-2, IL-3, IL-4, IL-5, IL-6, IL-7, IL-8, IL-9, IL-10, IL-11, IL-12, IL-13, IL-14 or IL-15, IL-16, IL-17, IL-18, IL-19, IL-20, IL-21, IL-22, IL-23, IL-24, IL-25) an interferon family member (alpha, beta or gamma), a CSF selected from the group consisting of G-CSF, GM-CSF, M-CSF, or GM-CSF/IL-3 fusions, or other growth factors such as SCF, EPO, leukemia inhibitory factor (“LWF”) or fibroblast growth factor (“FGF”).

Further embodiments include methods of using Flt3-L in autologous, allogeneic or syngeneic hematopoietic cell transplantation procedures incorporating nonmyeloablative conditioning regimens, comprising administering a Flt3-L composition to a patient in need of a hematopoietic cell transplant and transplanting hematopoietic cells to the patient, wherein the method further comprises the step of administering one or more additional cytokine/growth factors, wherein the growth factor is selected from the group consisting of an interleukin family member (e.g., IL-1, IL-2, IL-3, IL-4, IL-5, IL-6, IL-7, IL-8, IL-9, IL-10, IL-11, IL-12, IL-13, IL-14 or IL-15, IL-16, IL-17, IL-18, IL-19, IL-20, IL-21, IL-22, IL-23, IL-24, IL-25) an interferon family member (alpha, beta or gamma), a CSF selected from the group consisting of G-CSF, GM-CSF, M-CSF, or GM-CSF/IL-3 fusions, or other growth factors such as SCF, EPO, leukemia inhibitory factor-(“LIF”) or fibroblast growth factor (“FGF”). The Flt3-L and optional cytokine/growth factor(s) may be administered prior to, concurrent with and/or subsequent to the nonmyeloablative conditioning regimen and/or transplanting the hematopoietic cells.

A further embodiment includes method for conducting autologous, allogeneic or syngeneic hematopoietic cell transplantation, comprising: (1) collecting hematopoietic cells, such as, but not limited to, progenitor cells or stem cells, from a patient prior to cytoreductive therapy; (2) expanding the hematopoietic cells ex vivo with Flt3-L to provide a cellular preparation comprising increased numbers of hematopoietic cells; and (3) administering the cellular preparation to the patient in conjunction with or following some form of a nonmyeloablative conditioning regimen. A further embodiment includes using hematopoietic cells that have not been expanded ex vivo, such as autologous that have been generated in vivo after administration of one or more cytokines and/or hematopoietic growth factors. Hematopoietic cells may be obtained from peripheral blood harvests or bone marrow explants. Optionally, one or more cytokines/growth factors, selected from the group consisting an interleukin family member (e.g., IL-1, IL-2, IL-3, IL-4, IL-5, IL-6, IL-7, IL-8, IL-9, IL-10, IL-11, IL-12, IL-13, IL-14 or IL-15, IL-16, IL-17, IL-18, IL-19, IL-20, IL-21, IL-22, IL-23, IL-24, IL-25) an interferon family member (alpha, beta or gamma), a CSF selected from the group consisting of G-CSF, GM-CSF, M-CSF, or GM-CSF/IL-3 fusions, or other growth factors such as SCF, EPO, leukemia inhibitory factor (“LIF”) or fibroblast growth factor (“FGF”) can be administered to aid in the proliferation of particular hematopoietic cell types or affect the cellular function of the resulting proliferated hematopoietic cell population. The transplantation method described immediately above optionally comprises a preliminary in vivo procedure comprising administering Flt3-L alone or in sequential or concurrent combination with one or more cytokines/growth factors to a patient to recruit the hematopoietic cells into peripheral blood prior to their harvest. Suitable cytokines/growth factors are listed above. The transplantation method described immediately above optionally comprises a subsequent in vivo procedure comprising administering Flt3-L alone or in sequential or concurrent combination with an engraftment growth factor to a patient following transplantation of the cellular preparation to facilitate engraftment and augment proliferation of engrafted hematopoietic progenitor or stem cells from the cellular preparation. Suitable engraftment factors may be selected from the cytokines/growth factors listed above, such as, but not limited to GM-CSF, G-CSF, IL-3, IL-1, EPO and GM-CSF/IL-3 fusions.

As described above, the methods for using Flt3-L compositions in hematopoietic cell transplantation procedures described herein include standard cytoreductive therapies administered in a nonmyeloablative conditioning regimen. This includes treating the patients in need of a hematopoietic cell transplant with standard radiation, chemotherapeutic agents, surgery and/or immunosuppressive agents in any combination and temporal arrangement. The type and dosing of radiation, chemotherapeutic agents and/or immunosuppressive agents are to be selected, administered and monitored by a qualified physician. The various forms of radiotherapy, chemotherapy and immunosuppression therapy are known in the art and one of skill in the art would readily employ these established practices to the methods described herein.

As described above, alternative embodiments include methods of treating patients in need of a hematopoietic cell transplant comprising administering a Flt3-L composition to the patient, administering a nonmyeloablative conditioning regimen, transplanting hematopoietic cells into the patient, and administering one or more immunosuppressive agents to the patient one or more times post-transplant as needed. It is understood that the immunosuppressive agents may be used in the nonmyeloablative conditioning regimen, as well as in post-transplant therapy.

Examples of immunosuppressive agents include, but are not limited to steroids, corticosteroids, Azathioprine, Mycophenolate Mofetil (MMF, CellCept®), Cyclosporine (CsA, Cyclosporine A, Sandimmune®, Neoral®), Tacrolimus (FK-506, Prograf®), Sirolimus, Everolimus, FTY720, OKT3 (huOKT3-gamma), OKT4, Dacilsumab, Basilixmab, Rituximab, Alemtuzumab, Lea294, Antithymocyte Globulin (ATG, ATG-E, ATG-R Atgam®, Thymoglobulin®), Fludarabine (2-FLAA, fludarabine phosphate, Fludara®), Busalfan (busulphan, Myleran®), Cidofovir (Vistide®), Ribavirin (RTCA, Tribavirin, Rebetol®, Virazole®), Campath-1H, FK778, Indolyl-ASC and the like. Of course, it is understood that the list of immunosuppressive agents is illustrative and is meant to exemplify individual agents that represent classes of agents. For example, when Dacilsumab and Basilixmab are recited, it is meant to also include other antibody-based immunosuppressive agents and so on.

Examples of oncology drugs that may be used in the methods described herein include, but are not limited to: Aldesleukin (Proleukin®); Alemtuzumab (Campath®); Alitretinoin (Panretin®); Allopurinol (Zyloprim®); Altretamine (Hexalen®); Amifostine (Ethyol®); Anastrozole (Arimidex®); Arsenic trioxide (Trisenox®); Asparaginase (Elspar®); Bexarotene (Targretin®); Bleomycin (Blenoxane®); Busulfan intravenous (Busulfex®); Busulfan oral (Myleran®); Calusterone (Methosarb®); Capecitabine (Xeloda®); Carboplatin (Paraplatin®); Carmustine (BCNU, BiCNU); Chlorambucil (Leukeran®); Cisplatin (Platinol®); Cladribine (Leustatin®, 2-CdA); Cyclophosphamide (Cytoxan®, Neosar®, injection or tablet); Cytarabine (Cytosar-U®); Cytarabine liposomal (DepoCyt®); Dacarbazine (DTIC-Dome®); Dactinomycin, actinomycin D (Cosmegen®); Darbepoetin alfa (Aranesp®); Daunorubicin liposomal (DanuoXome®); Daunorubicin, daunomycin (Daunorubicin®); Daunorubicin, daunomycin (Cerubidine®); Denileukin diftitox (Ontak®); Dexrazoxane (Zinecard®); Docetaxel (Taxotere®); Doxorubicin (Adriamycin®, Rubex®); Doxorubicin (Adriamycin PFS® Injection, intravenous injection); Doxorubicin liposomal (Doxil®); Dromostanolone proprionate (Dromostanolone®); Elliott's B Solution; Epirubicin (Ellence®); Epoetin alfa (Epogen®); Estramustine (Emcyt®); Etoposide phosphate (Etopophos®); Etoposide, VP-16 (Vepesid®); Exemestane (Aromrasin®); Filgrastim (Neupogen®); Floxuridine (FUDR®); Fludarabine (Fludara®); Fluorouracil, 5-FU (Adrucil®); Fulvestrant (Faslodex®); Gemcitabine (Gemzar®); Gemtuzumab ozogamicin (Mylotarg®); Goserelin acetate (Zoladex® and Zoladex Implant®); Hydroxyurea (Hydrea®); Ibritumomab Tiuxetan (Zevalin®); Idarubicin (Idamycin®); Ifosfamide (IFEX®); Imatinib mesylate (Gleevec®); Interferon alfa-2a (Roferon-A®); Interferon alfa-2b (Intron A®); Irinotecan (Camptosar®); Letrozole (Femara®); Leucovorin (Wellcovorin®, Leucovorin®); Levamisole (Ergamisol®); Lomustine, CCNU (CeeBU®); Meclorethamine, nitrogen mustard (Mustargen®); Megestrol acetate (Megace®); Melphalan, L-PAM (Alkeran®); Mercaptopurine, 6-MP (Purinethol®); Mesna Mesnex; Methotrexate; Methoxsalen (UVadex®) Mitomycin C (Mutamycin®); Mitomycin C (Mjtozytrex®); Mitotane (Lysodren®); Mitoxantrone (Novantrone®); Nandrolone phenpropionate (Durabolin-50®); Nofetumomab (Verluma®); Oprelvekin (Neumega®); Oxaliplatin (Eloxatin®); Paclitaxel (Paxene®); Paclitaxel (Taxol®); Pamidronate (Aredia®); Pegademase (Adagen®); Pegaspargase (Oncaspar®); Pegfilgrastim (Neulastav); Pentostatin (Nipent®); Pipobroman (Vercyte®); Plicamycin, mithramycin (Mithracin®); Porfimer sodium (Photofrin®); procarbazine (Matulane®); Quinacrine (Atabrine®); Rasburicase (Elitek®); Rituximab (Rituxan®); Sargramostim (Prokine®); Streptozocin (Zanosar®); Tamoxifen (Nolvadex®); Temozolomide (Temodar®); Teniposide, VM-26 (Vumon®); Testolactone (Teslac®); Thioguanine, 6-TG (Thioguanine®); Thiotepa (Thioplex®); Topotecan (Hycamtin®); Toremifene (Fareston®); Tositumomab (Bexxar®); Trastuzumab (Herceptin®); Tretinoin, ATRA (Vesanoid®); Uracil Mustard; Valrubicin (Valstar®); Vincristine (Oncovin®); Vinorelbine (Navelbine®); Zoledronate (Zometa®) and the like.

Flt3-L in hematopoietic cell transplantation procedures incorporating nonmyeloablative conditioning regimens may be used in combination with adoptive immunotherapy, such as but not limited to Donor Lymphocyte Infusions (DLI) (see for example, Kolb, H., et al., Blood 1995 86: 2041-2050; Slavin S, et al. Exp Hematol 1995; 23: 1553-1562 or S. Slavin, et al., Bone Marrow Transplant 2001 28: 795-798). Another example of adoptive immunotherapy is the use of tumor-specific T-cell clones. If a patient should relapse, i.e. the malignancy returns, one or a series of DLIs may be included in the methods described herein. For example, after administering a Flt3-L composition to a patient, subjecting the patient to a nonmyeloablative conditioning regimen, performing a hematopoietic cell transplant and optionally administering one or more immunsuppressive agents to the patient, one may further treat the patient with one or more DLIs. It is understood that the Flt3-L composition may be administered at any time during the course of treatment and follow-up care. It has been shown in the art that donor lymphocyte infusions to patients with leukemia who relapsed after an allograft can have complete remission of their malignancy due to the graft-vs-malignancy (GVM) effect. In a number of patients who relapse post BMT, remission may be accomplished with adoptive allogeneic cell-mediated immunotherapy mediated by donor lymphocyte infusion (DLI) (e.g., Slavin S, et al. Exp Hematol 1995; 23: 1553-1562). DLI is particularly effective in patients with relapsed chronic myeloid leukemia (CML). Since the graft-versus-leukemia (GVL) effects induced with DLI are mediated primarily by alloreactive donor T lymphocytes, activation of GVL effector cells in patients resistant to DLI can be accomplished by donor lymphocytes stimulated in vivo and/or in vitro with recombinant interleukin 2 (rIL-2).

The use of DLIs as an optional step in treatment methods using Flt3-L compositions, nonmyeloablative conditioning regimens and hematopoietic cell transplant is an attractive embodiment of the invention. Adoptive transfer of donor immunity to the host in the course of hematopoietic cell transplant is well established in experimental animals and man. The concept of using a similar principle for more effective immunotherapy of resistant malignancy, by using donor-derived allo-reactive lymphocytes is supported by data in preclinical animal models (Morecki S, Slavin S., J Hematother Stem Cell Res 2000; 9: 355-377). Data from preclinical animal models suggest that allogeneic lymphocytes may be effectively activated against allogeneic tumor cells of host origin, while in parallel down-regulating their alloreactive potential against normal host somatic cells. Such a procedure may represent one possible approach for accomplishing GVM effects independently of GVHD. The existence of GVM effects, in part independently of GVHD, was also documented in patients treated with DLI for relapse following bone marrow transplant Slavin, et al. describe treating relapsed leukemia with donor peripheral blood lymphocytes (PBL) pulsed in vitro against the patient's own alloantigens presented by parental lymphocytes that may have become more immunogenic in response to treatment with alpha interferon (∝IFN), suggesting that improved immunotherapy against resistant relapsed leukemia may be accomplished by using specifically immune donor lymphocytes and that using immune donor lymphocytes, effective GVL may be accomplished independently of GVHD (Slavin et al. Bone Marrow Transplantation 2001 28: 795-798). In short, Slavin et al. pulsed donor lymphocytes in vitro with a mixture of irradiated peripheral blood lymphocytes (PBL) obtained from both parents, in order to trigger alloactivation of donor lymphocytes against host alloantigens presented by parental cells, using as stimulating cells maternal PBL expressing the shared maternal haplotype and paternal PBL expressing the shared paternal haplotype of the patient. Full hematologic, cytogenetic and molecular remission was induced, independently of GVH, and persisted for more than 9 years.

Example 6 describes how the use of Flt3-L facilitates stable engraftment, chimerism and tolerance—it is a sound prediction that the addition of some form of adoptive immunotherapy, such as DLIs would be of further benefit to certain patient populations. Therefore, embodiments of the invention include administering Flt3-L in hematopoietic cell transplantation procedures incorporating nonmyeloablative conditioning regimens in combination with Donor Lymphocyte Infusions (DLI). The DLIs may be administered as frequently as needed and at any time point required to facilitate engraftment of the transplant.

B. Diseases and Conditions

Flt3-L compositions may be used in the various methods described herein to treat patients suffering from hematopoietic, lymphatic, hematologic, hematological malignancies, bone marrow, myelodysplastic, myeloproliferative, leukemic disease or disorder that adversely affects the patient. More specific examples include chronic myelocytic leukemia (CML), chronic lymphocytic leukemia (CLL), acute myelocytic leukemia (AML), multiple myeloma (MM), and non-Hodgkin lymphoma (NHL). Further examples include myelodysplastic syndrome, aplastic anemia and cancers, such as leukemia (in all its forms). Examples of myelodysplastic syndromes that may be treated by the methods described herein include, but are not limited to refractory anemia, refractory anemia with ringed sideroblasts, refractory anemia with excess blasts, refractory anemia with excess blasts in transformation, and chronic myelomonocytic leukemia.

Flt3-ligand compositions may be used in the various methods described herein to treat patients suffering from pancytopenia, polycythemia, preleukemia, hemolytic anemia, hypochromic anemia, macrocytic anemia, myelophthisic anemia, aplastic anemia, neonatal anemia, refractory anemia. Further examples include all forms of leukemia, B-cell leukemia, hairy-cell leukemia, mast-cell leukemia, plasmacytic leukemia, radiation-induced leukemia, subleukemic leukemia, acute lymphocytic leukemia (acute B-cell leukemia, CALLA-positive leukemia, acute L1 lymphocytic leukemia, acute L2 lymphocytic leukemia, mixed-cell leukemia, acute T-cell leukemia), chronic lymphocytic leukemia, T-cell leukemia, myeloid leukemia (chronic myeloid leukemia, Philadelphia-negative myeloid leukemia, Philadelphia-positive myeloid leukemia, acute myelomonocytic leukemia, acute nonlymphocytic leukemia), lymphoma (malignant histiocytosis, Hodgkin disease, immunoproliferative small intestinal disease, Letterer-Siwe disease, plasmacytoma, reticuloendotheliosis), non-Hodgkin lymphomas (B-cell lymphomas, diffuse lymphomas, follicular lymphomas, high-grade lymphoma, intermediate-grade lymphoma, low-grade lymphoma, large-cell lymphoma, mixed-cell lymphoma, small-cell lymphoma, T-cell lymphoma, undifferentiated lymphoma), diffuse lymphomas (diffuse large-cell lymphoma, immunoblastic large-cell lymphoma, lymphoblastic lymphoma, diffuse mixed-cell lymphoma, diffuse small cleaved-cell lymphoma, small lymphocytic lymphoma, small noncleaved-cell lymphoma), and myeloproliferative disorders (myelophthisic anemia, acute erythroblastic leukemia, leukemoid reaction, myelofibrosis, myeloid metaplasia, polycythemia vera, thrombocytosis).

In further examples, Flt3-ligand compositions may be used in the various methods described herein to treat patients suffering from immunologic diseases, such as but not limited to autoimmune diseases. This is based in part on the success seen in the allograft studies in Example 6. It is known in the art that hematopoietic cell transplantation is being increasingly utilized for the treatment of a whole spectrum of severe autoimmune diseases refractory to conventional therapy. It has been postulated that if immunosuppressive regimens can eliminate or effectively reduce the level of autoreactive T and B cells, then regeneration of de novo immunity even in the autologous setting may bypass the initial breakdown of self-tolerance and ensure prolonged disease remission. (Burt, R., et al., Bone Marrow Transplantation 2003 31: 521-524). Embodiments of the invention include the use of Flt3-L in hematopoietic cell transplants incorporating a nonmyeloablative conditioning regimen for the treatment of autoimmune diseases. The additional use of post-transplant immunosuppression and/or adoptive immunotherapy is also envisioned.

More specific examples of autoimmune diseases that may be treated with the methods described herein is provided in the table below.

Nervous System:
Multiple sclerosis
Myasthenia gravis
Autoimmune neuropathies
such as Guillain-Barré
Autoimmune uveitis
Blood:
Autoimmune hemolytic anemia
Pernicious anemia
Autoimmune thrombocytopenia
Blood Vessels:
Temporal arteritis
Anti-phospholipid syndrome
Vasculitides such as
Wegener's granulomatosis
Behcet's disease
Skin:
Psoriasis
Dermatitis herpetiformis
Pemphigus vulgaris
Vitiligo
Gastrointestinal System:
Crohn's Disease
Ulcerative colitis
Primary biliary cirrhosis
Autoimmune hepatitis
Endocrine Glands:
Type 1 or immune-mediated
diabetes mellitus
Grave's Disease
Hashimoto's thyroiditis
Autoimmune oophoritis and
orchitis
Autoimmune disease of the
adrenal gland
Multiple Organs Including the
Musculoskeletal System:*
Rheumatoid arthritis
Systemic lupus erythematosus
Scleroderma
Polymyositis, dermatomyositis
Spondyloarthropathies such as
ankylosing spondylitis
Sjogren's syndrome

In alternative embodiments, Flt3-L compositions in combination with hematopoietic cell transplantation and nonmyeloablative conditioning regimens (and optional post-transplant immunosuppression and/or adoptive immunotherapy may be used to treat solid tumors. This is based on the observation that cytokine-refractory metastatic renal cell carcinoma (RCC) may regress following allogeneic transplantation. Pilot trials and recent in vitro data have provided clear evidence that the graft-versus-tumor effect mounted against RCC can produce clinically meaningful regression of a metastatic solid tumor. Given this observation, one can reasonably speculate that the use of Flt3-L in conjunction with nonmyeloablative hematopoietic cell transplants may be efficacious in the treatment of cancers that are refractory to conventional therapy, such as genitourinary tumors, including metastatic bladder and prostate cancer. (see, Drachenberg D, Childs R W., Urol Clin North Am. 2003 August;30(3):611-22). Therefore, embodiments of the invention also include treating cancers of any and all organs of the body. Some examples include, but are not limited to: mammalian sarcomas and carcinomas, such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma and the like.

II. Flt3-Ligand

A. Biological Activity

Flt3-ligand (Flt3-Ligand and Flt3-L are used interchangeably and have the same meaning) affects the growth of pluripotent hematopoietic stem and progenitor cells, as well as a number of lineages in the lymphoid and myeloid pathways. A synergistic effect with a wide range of colony stimulating factors, interleukins and soluble thrombopoietin, to promote growth and colony formation of committed and primitive progenitor cells has been demonstrated. In vivo administration of Flt3-ligand to mice results in a significant expansion of hematopoietic progenitor cells. In particular, Flt3-ligand causes a significant increase in the number of progenitors in the bone marrow (5-fold) and spleen (100-fold), as well as increasing the number of immature B cells in these tissues. A 200-500 fold increase in the number of hematopoietic progenitor cells has been reported in the peripheral blood following treatment. Flt3-ligand alone and in combination with other cytokines (IL-3, IL-6 or IL-17) has been shown to preferentially stimulate T cell development from the most primitive thymic progenitor cells. Additionally, in vitro studies have demonstrated that Flt3-ligand can induce the expansion of fetal liver, bone marrow or thymic natural killer (NK) cell progenitors, as well as costimulate (with IL-15 alone or a combination of IL-6/IL-7/IL-15) the generation of CD56+ NK cells from their progenitors. Flt3-ligand has also been shown to increase NK cell activity, NK cell proliferative responses, and generation of lymphocyte activated killer (LAK) cells, suggesting a potential role for Flt3-ligand in anti-cancer and anti-viral therapy. For a review of Flt3-ligand see “Flt3-ligand and Its Influence on Immune Reactivity” Cytokine, vol.12, no. 2, pp 97-100 (2000).

Administration of Flt3-ligand (both in vivo and in vitro) causes targeted expansion of hematopoietic stem and progenitor cells resulting in a generalized expansion of dendritric cells (DC) in multiple tissue sites. Dendritic cells comprise a heterogeneous cell population with distinctive morphology and a widespread tissue distribution. The dendritic cell system and its role in immunity are reviewed by Steinman, R. M., Annu. Rev. Immunol., 9:271-296 (1991), and is incorporated herein by reference. Dendritic cells have a high capacity for sensitizing MHC-restricted T cells and are very effective at presenting antigens to T cells in situ, both self-antigens during T cell development and tolerance and foreign antigens during immunity.

As used herein, a dendritic cell, or DC, refers to any member of a diverse population of morphologically similar cell types found in lymphoid or non-lymphoid tissues. DCs are a class of “professional” antigen presenting cells, and have a high capacity for sensitizing MHC-restricted T cells. Depending upon their lineage and stage of maturation, DCs may be recognized by function, or by phenotype, particularly by cell surface phenotype. These cells are characterized by their distinctive morphology, phagocytic/endocytotic capacity, high levels of surface MHC-class II expression and ability to present antigen to T cells, particularly to naive T cells (Banchereau, et al., Annu. Rev. Immunol., 18:767-811, 2000 and U.S. Pat. No. 6,274,378, incorporated herein by reference for its description of such cells). For illustrative purposes only, DCs described herein may be characterized by veil-like projections and expression of the cell surface markers CD1a⁺, CD4⁺, CD86⁺, or HLA-DR⁺. Mature DCs are typically CD11c⁺, while precursors of DCs include those having the phenotype CD11c⁻, IL-3Rα^{low}; and those that are CD11c⁻ IL-3Rα^{high}. Treatment with GM-CSF in vivo preferentially expands CD11b^{high}, CD11c^{high} DC in mice, while Flt3-ligand has been shown to expand CD11c⁺ IL-3Rα^{low} DC, and CD11c⁻ IL-3Rα^{high} DC precursors in humans. Functionally, dendritic cells maybe identified by any convenient assay for determination of antigen presentation. Such assays may include testing the ability to stimulate antigen-primed or naive T cells by presentation of a test antigen, following by determination of T cell proliferation, release of IL-2, and the like.

B. Flt3-Ligand Compositions

Flt3-L can be used to prepare Flt3-L compositions, such as pharmaceutical compositions, to be used in allogeneic, syngeneic or autologous transplantation methods. Particular embodiments of the present invention include the use of Flt3-L in autologous, allogeneic or syngeneic hematopoietic cell transplantation methods.

As used herein, the term Flt3-ligand refers to a genus of polypeptides that are described in U.S. Pat. Nos. 5,554,512, 6,291,661 and 6,632,424 which are incorporated herein by reference. Forms of Flt3-ligand that may be used in the methods described herein include, but are not limited to, murine and human Flt3-ligand. A human Flt3-ligand cDNA was deposited with the American Type Culture Collection, Rockville, Md., USA (ATCC) on Aug. 6, 1993 and assigned accession number ATCC 69382 and a mouse Flt3-ligand cDNA was deposited on the same day and assigned accession number ATCC 69286. The deposits were made under the terms of the Budapest Treaty. Flt3-ligand is commercially available from Immunex Corporation, Seattle, Wash. Flt3-ligand can be made according to the methods described in the documents cited above. Flt3-ligand may be modified by the addition of one or more water-soluble polymers, such as, but not limited to, polyethylene glycol to increase bio-availability and/or pharmacokinetic half-life. In alternative embodiments, Flt3-binding proteins that mimic the biological effects of Flt3-ligand may be used in the immunization protocols described herein. For example, WO 95/27062 describes agonistic antibodies to Flt3, the receptor for Flt3-ligand, from which various Flt3 binding proteins can be prepared.

Additional embodiments of Flt3-ligand are biologically active, soluble forms of Flt3-ligand, and particularly those forms comprising the extracellular domain or one or more fragments of the extracellular domain. Soluble forms of Flt3-ligand are polypeptides that are capable of being secreted from the cells in which they are expressed. In such forms part or all of the intracellular and transmembrane domains of the polypeptide are deleted such that the polypeptide is fully secreted from the cell in which it is expressed. The intracellular and transmembrane domains of polypeptides of the invention can be identified in accordance with known techniques for determination of such domains from sequence information. Soluble Flt3-ligand also includes those polypeptides which include part of the transmembrane region, provided that the soluble Flt3-ligand is capable of being secreted from a cell, and preferably retains the capacity to bind the Flt3 receptor and effectuate its biological effects. Soluble Flt3-ligand further includes oligomers or fusion polypeptides-comprising the extracellular portion of at least one Flt3-ligand polypeptide, and fragments of any of these polypeptides that have Flt3-ligand polypeptide activity.

Human Flt3-ligand may comprise an amino acid sequence selected from the group consisting of amino acids 28 to Xaa of SEQ ID NO:6, wherein Xaa is an amino acid from 160 to 235. Alternative embodiments comprise an amino acid sequence selected from the group consisting of amino acids 27 to Xaa of SEQ ID NO:6, wherein Xaa is an amino acid from 160 to 235. Murine Flt3-ligand may comprise an amino acid sequence selected from the group consisting of amino acids 28 to Yaa of SEQ ID NO:2, wherein Yaa is an amino acid from 163 to 231. Embodiments of soluble human Flt3-ligand include: the amino acid sequence of residues 27-160 of SEQ ID NO:6 (inclusive), 28-160 of SEQ ID NO:6 (inclusive), 27-179 SEQ.ID NO:6 (inclusive), 27-182 SEQ ID NO:6 (inclusive), 28-182 of SEQ ID NO:6 (inclusive), 27-235 SEQ ID NO:6 (inclusive) and 28-235 of SEQ ID NO:6 (inclusive). Embodiments of soluble murine Flt3-ligand include: the amino acid sequence of residues 28-163 of SEQ ID NO:2 (inclusive), the amino acid sequence of residues 28-188 of SEQ ID NO:2 (inclusive) and the amino acid sequence of residues 28-231 of SEQ ID NO:2 (inclusive).

The term “biologically active” as it refers to flt3-L, means that the flt3-L is capable of binding to flt3. Alternatively, “biologically active” means the flt3-L is capable of transducing a stimulatory signal to the cell through the membrane-bound flt3.

“Isolated” means that flt3-L is free of association with other proteins or polypeptides, for example, as a purification product of recombinant host cell culture or as a purified extract.

Of course, Flt3-ligand variants that are substantially similar and retain comparable biological activity may be used in the methods described herein. The term “substantially similar” means a variant amino acid sequence that is at least 80% identical to a native amino acid sequence, or at least 90% identical. The percent identity of two amino acid or two nucleic acid sequences can be determined by visual inspection and mathematical calculation, or more preferably, the comparison is done by comparing sequence information using a computer program. An exemplary, preferred computer program is the Genetics Computer Group (GCG; Madison, Wis.) Wisconsin package version 10.0 program, ‘GAP’ (Devereux et al., 1984, Nucl. Acids Res. 12: 387). The preferred default parameters for the ‘GAP’ program includes: (1) The GCG implementation of a unary comparison matrix (containing a value of 1 for identities and 0 for non-identities) for nucleotides, and the weighted amino acid comparison matrix of Gribskov and Burgess, Nucl. Acids Res. 14:6745, 1986, as described by Schwartz and Dayhoff, eds., Atlas of Polypeptide Sequence and Structure, National Biomedical Research Foundation, pp. 353-358, 1979; or other comparable comparison matrices; (2) a penalty of 30 for each gap and an additional penalty of 1 for each symbol in each gap for amino acid sequences, or penalty of 50 for each gap-and an additional penalty of 3 for each symbol in each gap for nucleotide sequences; (3) no penalty for end gaps; and (4) no maximum penalty for long gaps. Other programs used by those skilled in the art of sequence comparison can also be used, such as, for example, the BLASTN program version 2.0.9, available for use via the National Library of Medicine website www.ncbi.nlm.nih.gov/gorf/wblast2.cgi, or the UW-BLAST 2.0 algorithm. Standard default parameter settings for UW-BLAST 2.0 are described at the following Internet site: sapiens.wustl.edu/blast/blast/#Features. In addition, the BLAST algorithm uses the BLOSUM62 amino acid scoring matrix, and optional parameters that can be used are as follows: (A) inclusion of a filter to mask segments of the query sequence that have low compositional complexity (as determined by the SEG program of Wootton and Federhen (Computers and Chemistry, 1993); also see Wootton and Federhen, 1996, Analysis of compositionally biased regions in sequence databases, Methods Enzymol. 266: 554-71) or segments consisting of short-periodicity internal repeats (as determined by the XNU program of Clayerie and States (Computers and Chemistry, 1993)), and (B) a statistical significance threshold for reporting matches against database sequences, or E-score (the expected probability of matches being found merely by chance, according to the stochastic model of Karlin and Altschul (1990); if the statistical significance ascribed to a match is greater than this E-score threshold, the match will not be reported.); preferred E-score threshold values are 0.5, or in order of increasing preference, 0.25, 0.1, 0.05, 0.01, 0.001, 0.0001, 1e-5, 1e-10, 1e-15, 1e-20, 1e-25, 1e-30, 1e-40, 1e-50, 1e-75, or 1e-100.

Flt3-ligand variants may comprise conservatively substituted sequences, meaning that a given amino acid residue is replaced by a residue having similar physiochemical characteristics. Examples of conservative substitutions include substitution of one aliphatic residue for another, such as Ile, Val, Leu, or Ala for one another, or substitutions of one polar residue for another, such as between Lys and Arg; Glu and Asp; or Gln and Asn. Other such conservative substitutions, for example, substitutions of entire regions having similar hydrophobicity characteristics, are well known. Naturally occurring variants are also encompassed by the invention. Examples of such variants are proteins that result from alternate mRNA splicing events or from proteolytic cleavage of the native protein, wherein the native biological property is retained.

Further modifications in the Flt3-ligand peptide or Flt3-ligand DNA sequences can be made by those skilled in the art using known techniques. Modifications of interest in the polypeptide sequences can include the alteration, substitution, replacement, insertion or deletion of a selected amino acid. For example, one or more of the cysteine residues can be deleted or replaced with another amino acid to alter the conformation of the molecule, an alteration which may involve preventing formation of incorrect intramolecular disulfide bridges upon folding or renaturation. Techniques for such alteration, substitution, replacement, insertion or deletion are well known to those skilled in the art (see, e.g., U.S. Pat. No. 4,518,584). As another example, N-glycosylation sites in the Flt3-ligand extracellular domain can be modified to preclude glycosylation, allowing expression of a reduced carbohydrate analog in mammalian and yeast expression systems. N-glycosylation sites in eukaryotic polypeptides are characterized by an amino acid triplet Asn-X-Y, wherein X is any amino acid except Pro and Y is Ser or Thr. Appropriate substitutions, additions, or deletions to the nucleotide sequence encoding these triplets will result in prevention of attachment of carbohydrate residues at the Asn side chain. Alteration of a single nucleotide, chosen so that Asn is replaced by a different amino acid, for example, is sufficient to inactivate an N-glycosylation site. Alternatively, the Ser or Thr can by replaced with another amino acid, such as Ala. Known procedures for inactivating N-glycosylation sites in polypeptides include those described in U.S. Pat. No. 5,071,972 and EP 276,846. Additional variants within the scope of the invention include polypeptides that can be modified to create derivatives thereof by forming covalent or aggregative conjugates with other chemical moieties, such as glycosyl groups, lipids, phosphate, acetyl groups and the like. Covalent derivatives can be prepared by linking the chemical moieties to functional groups on amino acid side chains or at the N-terminus or C-terminus of a polypeptide. Preferably, such alteration, substitution, replacement, insertion or deletion does not diminish the biological activity of Flt3-ligand. One example is a variant that binds with essentially the same binding affinity as does the native form. Binding affinity can be measured by conventional procedures, e.g., as described in U.S. Pat. No. 5,512,457 and as set forth herein.

Additional Flt3-ligand derivatives include covalent or aggregative conjugates of the polypeptides with other polypeptides or polypeptides, such as by synthesis in recombinant culture as N-terminal or C-terminal fusions. Examples of fusion polypeptides are discussed below in connection with oligomers. Further, fusion polypeptides can comprise peptides added to facilitate purification and identification. Such peptides include, for example, poly-His or the antigenic identification peptides described in U.S. Pat. No. 5,011,912 and in Hopp et al., Bio/Technology 6:1204, 1988. One such peptide is the FLAG® peptide, which is highly antigenic and provides an epitope reversibly bound by a specific monoclonal antibody, enabling rapid assay and facile purification of expressed recombinant polypeptide. A murine hybridoma designated 4E11 produces a monoclonal antibody that binds the FLAG® peptide in the presence of certain divalent metal cations, as described in U.S. Pat. No. 5,011,912. The 4E11 hybridoma cell line has been deposited with the American Type Culture Collection under accession no. HB 9259. Monoclonal antibodies that bind the FLAG® peptide are available from Eastman Kodak Co., Scientific Imaging Systems Division, New Haven, Conn.

Additional embodiments of Flt3-ligand that may be used in the methods described herein include oligomers or fusion polypeptides that contain a Flt3-ligand, one or more fragments of Flt3-ligand, or any of the derivative or variant forms of Flt3-ligand as disclosed herein, as well as in the U.S. patents listed above. In particular embodiments, the oligomers comprise soluble Flt3-ligand polypeptides. Oligomers can be in the form of covalently linked or non-covalently-linked multimers, including dimers, trimers, or higher oligomers. In an alternative embodiments, Flt3-ligand oligomers comprise multiple Flt3-ligand polypeptides joined via covalent or non-covalent interactions between peptide moieties fused to the polypeptides, such peptides having the property of promoting oligomerization. Leucine zippers and certain polypeptides derived from antibodies are among the peptides that can promote oligomerization of the polypeptides attached thereto, as described in more detail below.

Immunoglobulin-based Oligomers. Soluble Flt3-ligand and fragments thereof can be fused directly or through linker sequences to the Fc portion of an immunoglobulin. For a bivalent form of Flt3-ligand, such a fusion could be to the Fc portion of an IgG molecule. Other immunoglobulin isotypes can also be used to generate such fusions. For example, a polypeptide-IgM fusion would generate a decavalent form of the polypeptide of the invention. The term “Fc polypeptide” as used herein includes native and mutein forms of polypeptides made up of the Fc region of an antibody comprising any or all of the CH domains of the Fc region. Truncated forms of such polypeptides containing the hinge region that promotes dimerization are also included. Preferred Fc polypeptides comprise an Fc polypeptide derived from a human IgG1 antibody. As one alternative, an oligomer is prepared using polypeptides derived from immunoglobulins. Preparation of fusion polypeptides comprising certain heterologous polypeptides fused to various portions of antibody-derived polypeptides (including the Fc domain) has been described, e.g., by Ashkenazi et al. (PNAS USA 88:10535, 1991); Byrn et al. (Nature 344:677, 1990); and Hollenbaugh and Aruffo (“Construction of Immunoglobulin Fusion Polypeptides”, in Current Protocols in Immunology, Suppl. 4, pages 10.19.1-10.19.11, 1992). Methods for preparation and use of immunoglobulin-based oligomers are well known in the art. One embodiment of Flt3-ligand is directed to a dimer comprising two fusion polypeptides created by fusing a Flt3-ligand to an Fc polypeptide derived from an antibody. A gene fusion encoding the Flt3-ligand/Fc fusion polypeptide is inserted into an appropriate expression vector. Flt3-ligand/Fc fusion polypeptides are expressed in host cells transformed with the recombinant expression vector, and allowed to assemble much like antibody molecules, whereupon interchain disulfide bonds form between the Fc moieties to yield divalent molecules. One suitable Fc polypeptide, described in PCT application WO 93/10151, is a single chain polypeptide extending from the N-terminal hinge region to the native C-terminus of the Fc region of a human IgG1 antibody. Another useful Fc polypeptide is the Fc mutein described in U.S. Pat. No. 5,457,035 and in Baum et al., (EMBO J. 13:3992-4001, 1994). The amino acid sequence of this mutein is identical to that of the native Fc sequence presented in WO 93/10151, except that amino acid 19 has been changed from Leu to Ala, amino acid 20 has been changed from Leu to Glu, and amino acid 22 has been changed from Gly to Ala. The mutein exhibits reduced affinity for Fc receptors. The above-described fusion polypeptides comprising Fc moieties (and oligomers formed therefrom) offer the advantage of facile purification by affinity chromatography over Polypeptide A or Polypeptide G columns. In other embodiments, the polypeptides of the invention can be substituted for the variable portion of an antibody heavy or light chain. If fusion polypeptides are made with both heavy and light chains of an antibody, it is possible to form an oligomer with as many as four Flt3-ligand extracellular regions.

Peptide-linker Based Oligomers. Alternatively, the oligomer is a fusion polypeptide comprising multiple Flt3-ligand polypeptides, with or without peptide linkers (spacer peptides). Among the suitable peptide linkers are those described in U.S. Pat. Nos. 4,751,180 and 4,935,233. A DNA sequence encoding a desired peptide linker can be inserted between, and in the same reading frame as, the DNA sequences of the invention, using any suitable conventional technique. For example, a chemically synthesized oligonucleotide encoding the linker can be ligated between the sequences. In particular embodiments, a fusion polypeptide comprises from two to four soluble Flt3-ligand polypeptides, separated by peptide linkers. Suitable peptide linkers, their combination with other polypeptides, and their use are well known by those skilled in the art.

Leucine-Zippers. Another method for preparing the oligomers of Flt3-ligand involves use of a leucine zipper. Leucine zipper domains are peptides that promote oligomerization of the polypeptides in which they are found. Leucine zippers were originally identified in several DNA-binding polypeptides (Landschulz et al., Science 240:1759, 1988), and have since been found in a variety of different polypeptides. Among the known leucine zippers are naturally occurring peptides and derivatives thereof that dimerize or trimerize. The zipper domain (also referred to herein as an oligomerizing, or oligomer-forming, domain) comprises a repetitive heptad repeat, often with four or five leucine residues interspersed with other amino acids. Use of leucine zippers and preparation of oligomers using leucine zippers are well known in the art.

An aspect of the invention is soluble flt3-L polypeptides. Soluble flt3-L polypeptides comprise all or part of the extracellular domain of a native flt3-L but lack the transmembrane region that would cause retention of the polypeptide on a cell membrane. Soluble flt3-L polypeptides advantageously comprise the native (or a heterologous) signal peptide when initially synthesized to promote secretion, but the signal peptide is cleaved upon secretion of flt3-L from the cell. Soluble flt3-L polypeptides encompassed by the invention retain the ability to bind the flt3 receptor. Indeed, soluble flt3-L may also include part of the transmembrane region or part of the cytoplasmic domain or other sequences, provided that the soluble flt3-L protein can be secreted.

Soluble flt3-L may be identified (and distinguished from its non-soluble membrane-bound counterparts) by separating intact cells which express the desired protein from the culture medium, e.g., by centrifugation, and assaying the medium (supernatant) for the presence of the desired protein. The presence of flt3-L in the medium indicates that the protein was secreted from the cells and thus is a soluble form of the desired protein.

Soluble forms of flt3-L possess many advantages over the native bound flt3-L protein. Purification of the proteins from recombinant host cells is feasible, since the soluble proteins are secreted from the cells. Further, soluble proteins are generally more suitable for intravenous administration.

In one embodiment of the invention, soluble flt3-L was expressed as a fusion protein comprising (from N- to C-terminus) the yeast a factor signal peptide, a FLAG® peptide described below and in U.S. Pat. No. 5,011,912, and soluble flt3-L consisting of amino acids 28 to 188 of SEQ ID NO:2. This recombinant fusion protein is expressed in and secreted from yeast cells. The FLAG® peptide facilitates purification of the protein, and subsequently may be cleaved from the soluble flt3-L using bovine mucosal enterokinase. Isolated DNA sequences encoding soluble flt3-L proteins are encompassed by the invention.

Truncated flt3-L, including soluble polypeptides, may be prepared by any of a number of conventional techniques. A desired DNA sequence may be chemically synthesized using techniques known per se. DNA fragments also may be produced by restriction endonuclease digestion of a full length cloned DNA sequence, and isolated by electrophoresis on agarose gels. Linkers containing restriction endonuclease cleavage site(s) may be employed to insert the desired DNA fragment into an expression vector, or the fragment may be digested at cleavage sites naturally present therein. The well known polymerase chain reaction procedure also may be employed to amplify a DNA sequence encoding a desired protein fragment. As a further alternative, known mutagenesis techniques may be employed to insert a stop codon at a desired point, e.g., immediately downstream of the codon for the last amino acid of the extracellular domain.

In another approach, enzymatic treatment (e.g., using Bal 31 exonuclease) may be employed to delete terminal nucleotides from a DNA fragment to obtain a fragment having a particular desired terminus. Among the commercially available linkers are those that can be ligated to the blunt ends produced by Bal 31 digestion, and which contain restriction endonuclease cleavage site(s). Alternatively, oligonucleotides that reconstruct the N- or C-terminus of a DNA fragment to a desired point may be synthesized and ligated to the DNA fragment. The synthesized oligonucleotide may contain a restriction endonuclease cleavage site upstream of the desired coding sequence and position an initiation codon (ATG) at the N-terminus of the coding sequence.

As stated above, the invention provides isolated or homogeneous flt3-L polypeptides, both recombinant and non-recombinant. Variants and derivatives of native flt3-L proteins that retain the desired biological activity (e.g., the ability to bind flt3) may be obtained by mutations of nucleotide sequences coding for native flt3-L polypeptides. Alterations of the native amino acid sequence may be accomplished by any of a number of conventional methods. Mutations can be introduced at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion.

Alternatively, oligonucleotide-directed site-specific mutagenesis procedures can be employed to provide an altered gene wherein predetermined codons can be altered by substitution, deletion or insertion. Exemplary methods of making the alterations set forth above are disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); Kunkel (Proc. Natl. Acad. Sci. USA 82:488, 1985); Kunkel et al. (Methods in Enzymol. 154:367, 1987); and U.S. Pat. Nos. 4,518,584 and 4,737,462 all of which are incorporated by reference.

Flt3-L may be modified to create flt3-L derivatives by forming covalent or aggregative conjugates with other chemical moieties, such as glycosyl groups, lipids, phosphate, acetyl groups and the like. Covalent derivatives of flt3-L may be prepared by linking the chemical moieties to functional groups on flt3-L amino acid side chains or at the N-terminus or C-terminus of a flt3-L polypeptide or the extracellular domain thereof. Other derivatives of flt3-L within the scope of this invention include covalent or aggregative conjugates of flt3-L or its fragments with other proteins or polypeptides, such as by synthesis in recombinant culture as N-terminal or C-terminal fusions. For example, the conjugate may comprise a signal or leader polypeptide sequence (e.g. the α-factor leader of Saccharomyces) at the N-terminus of a flt3-L polypeptide. The signal or leader peptide co-translationally or post-translationally directs transfer of the conjugate from its site of synthesis to a site inside or outside of the cell membrane or cell wall.

Flt3-L polypeptide fusions can comprise peptides added to facilitate purification and identification of flt3-L. Such peptides include, for example, poly-His or the antigenic identification peptides described in U.S. Pat. No. 5,011,912 and in Hopp et al., Bio/Technology 6:1204, 1988.

The invention further includes flt3-L polypeptides with or without associated native-pattern glycosylation. Flt3-L expressed in yeast or mammalian expression systems (e.g., COS-7 cells) may be similar to or significantly different from a native flt3-L polypeptide in molecular weight and glycosylation pattern, depending upon the choice of expression system. Expression of flt3-L polypeptides in bacterial expression systems, such as E. coli, provides non-glycosylated molecules.

Equivalent DNA constructs that encode various additions or substitutions of amino acid residues or sequences, or deletions of terminal or internal residues or sequences not needed for biological activity or binding are encompassed by the invention. For example, N-glycosylation sites in the flt3-L extracellular domain can be modified to preclude glycosylation, allowing expression of a reduced carbohydrate analog in mammalian and yeast expression systems. N-glycosylation sites in eukaryotic polypeptides are characterized by an amino acid triplet Asn-X-Y, wherein X is any amino acid except Pro and Y is Ser or Thr. The murine and human flt3-L proteins each comprise two such triplets, at amino acids 127-129 and 152-154 of SEQ ID NO:2, and at amino acids 126-128 and 150-152 of SEQ ID NO:6, respectively. Appropriate substitutions, additions or deletions to the nucleotide sequence encoding these triplets will result in prevention of attachment of carbohydrate residues at the Asn side chain. Alteration of a single nucleotide, chosen so that Asn is replaced by a different amino acid, for example, is sufficient to inactivate an N-glycosylation site. Known procedures for inactivating N-glycosylation sites in proteins include those described in U.S. Pat. No. 5,071,972 and EP 276,846, hereby incorporated by reference.

In another example, sequences encoding Cys residues that are not essential for biological activity can be altered to cause the Cys residues to be deleted or replaced with other amino acids, preventing formation of incorrect intramolecular disulfide bridges upon renaturation. Other equivalents are prepared by modification of adjacent dibasic amino acid residues to enhance expression in yeast systems in which KEX2 protease activity is present. EP 212,914 discloses the use of site-specific mutagenesis to inactivate KEX2 protease processing sites in a protein. KEX2 protease processing sites are inactivated by deleting, adding or substituting residues to alter Arg-Arg, Arg-Lys, and Lys-Arg pairs to eliminate the occurrence of these adjacent basic residues. Lys-Lys pairings are considerably less susceptible to KEX2 cleavage, and conversion of Arg-Lys or Lys-Arg to Lys-Lys represents a conservative and preferred approach to inactivating KEX2 sites. Both murine and human flt3-L contain two KEX2 protease processing sites at amino acids 216-217 and 217-218 of SEQ ID NO:2 and at amino acids 211-212 and 212-213 of SEQ ID NO:6, respectively.

Nucleic acid sequences within the scope of the invention include isolated DNA and RNA sequences that hybridize to the native flt3-L nucleotide sequences disclosed herein under conditions of moderate or severe stringency, and which encode biologically active flt3-L. Conditions of moderate stringency, as defined by Sambrook et al. Molecular Cloning: A Laboratory Manual, 2 ed. Vol. 1, pp. 1.101-104, Cold Spring Harbor Laboratory Press, (1989), include use of a prewashing solution of 5×SSC, 0.5% SDS, 1.0 mM EDTA (pH 8.0) and hybridization conditions of about 55° C., 5×SSC, overnight. Conditions of severe stringency include higher temperatures of hybridization and washing. The skilled artisan will recognize that the temperature and wash solution salt concentration may be adjusted as necessary according to factors such as the length of the probe.

Due to the known degeneracy of the genetic code wherein more than one codon can encode the same amino acid, a DNA sequence may vary from that shown in SEQ ID NO:1 and SEQ ID NO:5 and still encode an flt3-L protein having the amino acid sequence of SEQ ID NO:2 and SEQ ID NO:6, respectively. Such variant DNA sequences may result from silent mutations (e.g., occurring during PCR amplification), or may be the product of deliberate mutagenesis of a native sequence.

DNA that are equivalents to the DNA sequence of SEQ ID NO:1 or SEQ ID NO:5, will hybridize under moderately stringent conditions to the native DNA sequence that encode polypeptides comprising amino acid sequences of 28-163 of SEQ ID NO:2 or 28-160 of SEQ ID NO:6. Examples of flt3-L proteins encoded by such DNA, include, but are not limited to, flt3-L fragments (soluble or membrane-bound) and flt3-L proteins comprising inactivated N-glycosylation site(s), inactivated KEX2 protease processing site(s), or conservative amino acid substitution(s), as described above. Flt3-L proteins encoded by DNA derived from other mammalian species, wherein the DNA will hybridize to the cDNA of SEQ ID NO:1 or SEQ ID NO:5, are also encompassed.

Variants possessing the requisite ability to bind flt3 receptor may be identified by any suitable assay. Biological activity of flt3-L may be determined, for example, by competition for binding to the ligand binding domain of flt3 receptor (i.e. competitive binding assays).

One type of a competitive binding assay for a flt3-L polypeptide uses a radiolabeled, soluble human flt3-L and intact cells expressing cell surface flt3 receptors. Instead of intact cells, one could substitute soluble flt3 receptors (such as a flt3:Fc fusion protein) bound to a solid phase through the interaction of a Protein A, Protein G or an antibody to the flt3 or Fc portions of the molecule, with the Fc region of the fusion protein. Another type of competitive binding assay utilizes radiolabeled soluble flt3 receptors such as a flt3:Fc fusion protein, and intact cells expressing flt3-L. Alternatively, soluble flt3-L could be bound to a solid phase to positively select flt3 expressing cells.

Competitive binding assays can be performed following conventional methodology. For example, radiolabeled flt3-L can be used to compete with a putative flt3-L homolog to assay for binding activity against surface-bound flt3 receptors. Qualitative results can be obtained by competitive autoradiographic plate binding assays, or Scatchard plots may be utilized to generate quantitative results.

Alternatively, flt3-binding proteins, such as flt3-L and anti-flt3 antibodies, can be bound to a solid phase such as a column chromatography matrix or a similar substrate suitable for identifying, separating or purifying cells that express the flt3 receptor on their surface. Binding of flt3-binding proteins to a solid phase contacting surface can be accomplished by any means, for example, by constructing a flt3-L:Fc fusion protein and binding such to the solid phase through the interaction of Protein A or Protein G. Various other means for fixing proteins to a solid phase are well known in the art and are suitable for use in the present invention. For example, magnetic microspheres can be coated with flt3-binding proteins and held in the incubation vessel through a magnetic field. Suspensions of cell mixtures containing hematopoietic progenitor or stem cells are contacted with the solid phase that has flt3-binding proteins thereon. Cells having the flt3 receptor on their surface bind to the fixed flt3-binding protein and unbound cells then are washed away. This affinity-binding method is useful for purifying, screening or separating such flt3-expressing cells from solution. Methods of releasing positively selected cells from the solid phase are known in the art and encompass, for example, the use of enzymes. Such enzymes are preferably non-toxic and non-injurious to the cells and are preferably directed to cleaving the cell-surface binding partner. In the case of flt3:flt3-L interactions, the enzyme preferably would cleave the flt3 receptor, thereby freeing the resulting cell suspension from the “foreign” flt3-L material. The purified cell population then may be expanded ex vivo prior to transplantation to a patient in an amount sufficient to reconstitute the patient's hematopoietic and immune system.

Alternatively, mixtures of cells suspected of containing flt3+cells first can be incubated with a biotinylated flt3-binding protein. Incubation periods are typically at least one hour in duration to ensure sufficient binding to flt3. The resulting mixture then is passed through a column packed with avidin-coated beads, whereby the high affinity of biotin for avidin provides the binding of the cell to the beads. Use of avidin-coated beads is known in the art. See Berenson, et al. J. Cell. Biochem., 10D:239 (1986). Wash of unbound material and the release of the bound cells is performed using conventional methods.

In the methods described above, suitable flt3-binding proteins are flt3-L, anti-flt3 antibodies, and other proteins that are capable of high-affinity binding of flt3. A preferred flt3-binding protein is flt3-L.

As described above, flt3-L of the invention can be used to separate cells expressing flt3 receptors. In an alternative method, flt3-L or an extracellular domain or a fragment thereof can be conjugated to a detectable moiety such as ¹²⁵I to detect flt3 expressing cells. Radiolabeling with ¹²⁵I can be performed by any of several standard methodologies that yield a functional ¹²⁵I-flt3-L molecule labeled to high specific activity. Or an iodinated or biotinylated antibody against the flt3 region or the Fc region of the molecule could be used. Another detectable moiety such as an enzyme that can catalyze a colorimetric or fluorometric reaction, biotin or avidin may be used. Cells to be tested for flt3 receptor expression can be contacted with labeled flt3-L. After incubation, unbound labeled flt3-L is removed and binding is measured using the detectable moiety.

The binding characteristics of flt3-L (including variants) may also be determined using the conjugated, soluble flt3 receptors (for example, ¹²⁵I-flt3:Fc) in competition assays similar to those described above. In this case, however, intact cells expressing flt3 receptors, or soluble flt3 receptors bound to a solid substrate, are used to measure the extent to which a sample containing a putative flt3-L variant competes for binding with a conjugated a soluble flt3 to flt3-L.

Other means of assaying for flt3-L include the use of anti-flt3-L antibodies, cell lines that proliferate in response to flt3-L, or recombinant cell lines that express flt3 receptor and proliferate in the presence of flt3-L. For example, the BAF/BO3 cell line lacks the flt3 receptor and is IL-3 dependent. (See Hatakeyama, et al., Cell, 59: 837-845 (1989)). BAF/BO3 cells transfected with an expression vector comprising the flt3 receptor gene proliferate in response to either IL-3 or flt3-L. An example of a suitable expression vector for transfection of flt3 is the pCAV/NOT plasmid, see Mosley et al., Cell, 59: 335-348 (1989).

Flt3-L polypeptides may exist as oligomers, such as covalently-linked or non-covalently-linked dimers or trimers. Alternatively, oligomers may be linked by disulfide bonds formed between cysteine residues on different flt3-L polypeptides. In one embodiment of the invention; a flt3-L dimer is created by fusing flt3-L to the Fc region of an antibody (e.g., IgG1) in a manner that does not interfere with binding of flt3-L to the flt3-ligand-binding domain. The Fc polypeptide preferably is fused to the C-terminus of a soluble flt3-L (comprising only the extracellular domain). General preparation of fusion proteins comprising heterologous polypeptides fused to various portions of antibody-derived polypeptides (including the Fc domain) has been described, e.g., by Ashkenazi et al. (PNAS USA 88:10535, 1991) and Byrn et al. (Nature 344:677, 1990), hereby incorporated by reference. A gene fusion encoding the flt3-L:Fc fusion protein is inserted into an appropriate expression vector. Flt3-L:Fc fusion proteins are allowed to assemble much like antibody molecules, whereupon interchain disulfide bonds form between Fc polypeptides, yielding divalent flt3-L. If fusion proteins are made with both heavy and light chains of an antibody, it is possible to form a flt3-L oligomer with as many as four flt3-L extracellular regions. Alternatively, one can link two soluble flt3-L domains with a peptide linker.

Recombinant expression vectors containing a DNA encoding flt3-L can be prepared using well known methods. The expression vectors include a flt3-L DNA sequence operably linked to suitable transcriptional or translational regulatory nucleotide sequences, such as those derived from a mammalian, microbial, viral, or insect gene. Examples of regulatory sequences include transcriptional promoters, operators, or enhancers, an mRNA ribosomal binding site, and appropriate sequences which control transcription and translation initiation and termination. Nucleotide sequences are “operably linked” when the regulatory sequence functionally relates to the flt3-L DNA sequence. Thus, a promoter nucleotide sequence is operably linked to a flt3-L DNA sequence if the promoter nucleotide sequence controls the transcription of the flt3-L DNA sequence. The ability to replicate in the desired host cells, usually conferred by an origin of replication, and a selection gene by which transformants are identified, may additionally be incorporated into the expression vector.

In addition, sequences encoding appropriate signal peptides that are not naturally associated with flt3-L can be incorporated into expression vectors. For example, a DNA sequence for a signal peptide (secretory leader) may be fused in-frame to the flt3-L sequence so that flt3-L is initially translated as a fusion protein comprising the signal peptide. A signal peptide that is functional in the intended host cells enhances extracellular secretion of the flt3-L polypeptide. The signal peptide may be cleaved from the flt3-L polypeptide upon secretion of flt3-L from the cell.

Suitable host cells for expression of flt3-L polypeptides include prokaryotes, yeast or higher eukaryotic cells. Appropriate cloning and expression vectors for use with bacterial, fungal, yeast, and mammalian cellular hosts are described, for example, in Pouwels et al. Cloning Vectors: A Laboratory Manual, Elsevier, N.Y., (1985). Cell-free translation systems could also be employed to produce flt3-L polypeptides using RNAs derived from DNA constructs disclosed herein.

Prokaryotes include gram negative or gram positive organisms, for example, E. coli or Bacilli. Suitable prokaryotic host cells for transformation include, for example, E. coli, Bacillus subtilis, Salmonella typhimurium, and various other species within the genera Pseudomonas, Streptomyces, and Staphylococcus. In a prokaryotic host cell, such as E. coli, a flt3-L polypeptide may include an N-terminal methionine residue to facilitate expression of the recombinant polypeptide in the prokaryotic host cell. The N-terminal Met may be cleaved from the expressed recombinant flt3-L polypeptide.

Expression vectors for use in prokaryotic host cells generally comprise one or more phenotypic selectable marker genes. A phenotypic selectable marker gene is, for example, a gene encoding a protein that confers antibiotic resistance or that supplies an autotrophic requirement. Examples of useful expression vectors for prokaryotic host cells include those derived from commercially available plasmids such as the cloning vector pBR322 (ATCC 37017). pBR322 contains genes for ampicillin and tetracycline resistance and thus provides simple means for identifying transformed cells. To construct en expression vector using pBR322, an appropriate promoter and a flt3-L DNA sequence are inserted into the pBR322 vector. Other commercially available vectors include, for example, pKK223-3 (Pharmacia Fine Chemicals, Uppsala, Sweden) and pGEM1 (Promega Biotec, Madison, Wis., USA).

Promoter sequences commonly used for recombinant prokaryotic host cell expression vectors include β-lactamase (penicillinase), lactose promoter system (Chang et al., Nature 275:615, 1978; and Goeddel et al., Nature 281:544, 1979), tryptophan (trp) promoter system (Goeddel et al., Nucl. Acids Res. 8:4057, 1980; and EP-A-36776) and tac promoter (Maniatis, Molecular Cloning: A Laboratory Manual, Cold Spring Harbor Laboratory, p. 412, 1982). A particularly useful prokaryotic host cell expression system employs a phage λ P_{L} promoter and a cI857ts thermolabile repressor sequence. Plasmid vectors available from the American Type Culture Collection which incorporate derivatives of the λ P_{L} promoter include plasmid pHUB2 (resident in E. coli strain JMB9 (ATCC 37092)) and pPLc28 (resident in E. coli RR1 (ATCC 53082)).

Flt3-L polypeptides alternatively may be expressed in yeast host cells, preferably from the Saccharomyces genus (e.g., S. cerevisiae). Other genera of yeast, such as Pichia, K. lactis or Kluyveromyces, may also be employed. Yeast vectors will often contain an origin of replication sequence from a 21 yeast plasmid, an autonomously replicating sequence (ARS), a promoter region, sequences for polyadenylation, sequences for transcription termination, and a selectable marker gene. Suitable promoter sequences for yeast vectors include, among others, promoters for metallothionein, 3-phosphoglycerate kinase (Hitzeman et al., J. Biol. Chem. 255:2073, 1980) or other glycolytic enzymes (Hess et al., J. Adv. Enzyme Reg. 7:149, 1968; and Holland et al., Biochem. 17:4900, 1978), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Other suitable vectors and promoters for use in yeast expression are further described in Hitzeman, EPA-73,657 or in Fleer et. al., Gene, 107:285-195 (1991); and van den Berg et. al., Bio/Technology, 8:135-139 (1990). Another alternative is the glucose-repressible ADH2 promoter described by Russell et al. (J. Biol. Chem. 258:2674, 1982)-and Beier et al. (Nature 300:724, 1982). Shuttle vectors replicable in both yeast and E. coli may be constructed by inserting DNA sequences from pBR322 for selection and replication in E. coli (Amp^{r} gene and origin of replication) into the above-described yeast vectors.

The yeast α-factor leader sequence may be employed to direct secretion of the flt3-L polypeptide. The α-factor leader sequence is often inserted between the promoter sequence and the structural gene sequence. See, e.g., Kurjan et al., Cell 30:933, 1982; Bitter et al., Proc. Natl. Acad. Sci. USA 81:5330, 1984; U.S. Pat. No. 4,546,082; and EP 324,274. Other leader sequences suitable for facilitating secretion of recombinant polypeptides from yeast hosts are known to those of skill in the art. A leader sequence may be modified near its 3′ end to contain one or more restriction sites. This will facilitate fusion of the leader sequence to the structural gene.

Yeast transformation protocols are known to those of skill in the art. One such protocol is described by Hinnen et al., Proc. Natl. Acad. Sci. USA 75:1929, 1978. The Hinnen et al. protocol selects for Trp⁺ transformants in a selective medium, wherein the selective medium consists of 0.67% yeast nitrogen base, 0.5% casamino acids, 2% glucose, 10 μg/ml adenine and 20 μg/ml uracil.

Yeast host cells transformed by vectors containing ADH2 promoter sequence may be grown for inducing expression in a “rich” medium. An example of a rich medium is one consisting of 1% yeast extract, 2% peptone, and 1% glucose supplemented with 80 μg/ml adenine and 80 μg/ml uracil. Derepression of the ADH2 promoter occurs when glucose is exhausted from the medium.

Mammalian or insect host cell culture systems could also be employed to express recombinant flt3-L polypeptides. Baculovirus systems for production of heterologous proteins in insect cells are reviewed by Luckow and Summers, Bio/Technology 6:47 (1988). Established cell lines of mammalian origin also may be employed. Examples of suitable mammalian host cell lines include the COS-7 line of monkey kidney cells (ATCC CRL 1651) (Gluzman et al., Cell 23:175, 1981), L cells, C127 cells, 3T3 cells (ATCC CCL 163), Chinese hamster ovary (CHO) cells, HeLa cells, and BHK (ATCC CRL 10) cell lines, and the CV-1/EBNA-1 cell line derived from the African green monkey kidney cell line CVI (ATCC CCL 70) as described by McMahan et al. (EMBO J. 10: 2821, 1991).

Transcriptional and translational control sequences for mammalian host cell expression vectors may be excised from viral genomes. Commonly used promoter sequences and enhancer sequences are derived from Polyoma virus, Adenovirus 2, Simian Virus 40 (SV40), and human cytomegalovirus. DNA sequences derived from the SV40 viral genome, for example, SV40 origin, early and late promoter, enhancer, splice, and polyadenylation sites may be used to provide other genetic elements for expression of a structural gene sequence in a mammalian host cell. Viral early and late promoters are particularly useful because both are easily obtained from a viral genome as a fragment which may also contain a viral origin of replication (Fiers et al., Nature 273:113, 1978). Smaller or larger SV40 fragments may also be used, provided the approximately 250 bp sequence extending from the Hind In site toward the Bgl I site located in the SV40 viral origin of replication site is included.

Exemplary expression vectors for use in mammalian host cells can be constructed as disclosed by Okayama and Berg (Mol. Cell. Biol. 3:280, 1983). A useful system for stable high level expression of mammalian cDNAs in C127 murine mammary epithelial cells can be constructed substantially as described by Cosman et al. (Mol. Immunol. 23:935, 1986). A useful high expression vector, PMLSV N1/N4, described by Cosman et al., Nature 312:768, 1984 has been deposited as ATCC 39890. Additional useful mammalian expression vectors are described in EP-A-0367566, and in U.S. patent application Ser. No. 07/701,415, filed May 16, 1991, incorporated by reference herein. The vectors may be derived from retroviruses. In place of the native signal sequence, a heterologous signal sequence may be added, such as the signal sequence for IL-7 described in U.S. Pat. No. 4,965,195; the signal sequence for IL-2 receptor described in Cosman et al., Nature 312:768 (1984); the IL-4 signal peptide described in EP 367,566; the type I IL-1 receptor signal peptide described in U.S. Pat. No. 4,968,607; and the type II IL-1 receptor signal peptide described in EP 460,846.

Flt3-L as an isolated or homogeneous protein according to the invention may be produced by recombinant expression systems as described above or purified from naturally occurring cells. Flt3-L can be purified to substantial homogeneity, as indicated by a single protein band upon analysis by SDS-polyacrylamide gel electrophoresis (SDS-PAGE).

One process for producing flt3-L comprises culturing a host cell transformed with an expression vector comprising a DNA sequence that encodes flt3-L under conditions sufficient to promote expression of flt3-L. Flt3-L is then recovered from culture medium or cell extracts, depending upon the expression system employed. As is known to the skilled artisan, procedures for purifying a recombinant protein will vary according to such factors as the type of host cells employed and whether or not the recombinant protein is secreted into the culture medium.

For example, when expression systems that secrete the recombinant protein are employed, the culture medium first may be concentrated using a commercially available protein concentration filter, for example, an Amicon® or Millipore® Pellicon® ultrafiltration unit. Following the concentration step, the concentrate can be applied to a purification matrix such as a gel filtration medium. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Sulfopropyl groups are preferred. Finally, one or more reversed-phase high performance liquid chromatography (RP-HPLC) steps employing hydrophobic RP-HPLC media, (e.g., silica gel having pendant methyl or other aliphatic groups) can be employed to further purify flt3-L. Some or all of the foregoing purification steps, in various combinations, are well known and can be employed to provide a substantially homogeneous recombinant protein.

It is possible to utilize an affinity column comprising the ligand binding domain of flt3 receptors to affinity-purify expressed flt3-L polypeptides. Flt3-L polypeptides can be removed from an affinity column using conventional techniques, e.g., in a high salt elution buffer and then dialyzed into a lower salt buffer for use or by changing pH or other components depending on the affinity matrix utilized. Alternatively, the affinity column may comprise an antibody that binds flt3-L. Example 6 describes a procedure for employing flt3-L of the invention to generate monoclonal antibodies directed against flt3-L.

Recombinant protein produced in bacterial culture is usually isolated by initial disruption of the host cells, centrifugation, extraction from cell pellets if an insoluble polypeptide, or from the supernatant fluid if a soluble polypeptide, followed by one or more concentration, salting-out, ion exchange, affinity purification or size exclusion chromatography steps. Finally, RP-HPLC can be employed for final purification steps. Microbial cells can be disrupted by any convenient method, including freeze-thaw cycling, sonication, mechanical disruption, or use of cell lysing agents.

Transformed yeast host cells are preferably employed to express flt3-L as a secreted polypeptide in order to simplify purification. Secreted recombinant polypeptide from a yeast host cell fermentation can be purified by methods analogous to those disclosed by Urdal et al. (J. Chromatog. 296:171, 1984). Urdal et al. describe two sequential, reversed-phase HPLC steps for purification of recombinant human IL-2 on a preparative HPLC column.

Antisense or sense oligonucleotides comprising a single-stranded nucleic acid sequence (either RNA or DNA) capable of binding to a target flt3-L mRNA sequence (forming a duplex) or to the flt3-L sequence in the double-stranded DNA helix (forming a triple helix) can be made according to the invention. Antisense or sense oligonucleotides, according to the present invention, comprise a fragment of the coding region of flt3-L cDNA. Such a fragment generally comprises at least about 14 nucleotides, preferably from about 14 to about 30 nucleotides. The ability to create an antisense or a sense oligonucleotide, based upon a cDNA sequence for a given protein is described in, for example, Stein and Cohen, Cancer Res. 48:2659, 1988 and van der Krol et al., BioTechniques 6:958, 1988.

Binding of antisense or sense oligonucleotides to target nucleic acid sequences results in the formation of complexes that block translation (RNA) or transcription (DNA) by one of several means, including enhanced degradation of the duplexes, premature termination of transcription or translation, or by other means. The antisense oligonucleotides thus may be used to block expression of flt3-L proteins. Antisense or sense oligonucleotides further comprise oligonucleotides having modified sugar-phosphodiester backbones (or other sugar linkages, such as those described in WO91/06629) and wherein such sugar linkages are resistant to endogenous nucleases. Such oligonucleotides with resistant sugar linkages are stable in vivo (i.e., capable of resisting enzymatic degradation) but retain sequence specificity to be able to bind to target nucleotide sequences. Other examples of sense or antisense oligonucleotides include those oligonucleotides which are covalently linked to organic moieties, such as those described in WO 90/10448, and other moieties that increases affinity of the oligonucleotide for a target nucleic acid sequence, such as poly-(L-lysine). Further still, intercalating agents, such as ellipticine, and alkylating agents or metal complexes may be attached to sense or antisense oligonucleotides to modify binding specificities of the antisense or sense oliginucleotide for the target nucleotide sequence.

Antisense or sense oligonucleotides may be introduced into a cell containing the target nucleic acid sequence by any gene transfer method, including, for example, CaPO₄-mediated DNA transfection, electroporation, or by using gene transfer vectors such as Epstein-Barr virus. Antisense or sense oligonucleotides are preferably introduced into a cell containing the target nucleic acid sequence by insertion of the antisense or sense oligonucleotide into a suitable retroviral vector, then contacting the cell with the retrovirus vector containing the inserted sequence, either in vivo or ex vivo. Suitable retroviral vectors include, but are not limited to, the murine retrovirus M-MuLV, N2 (a retrovirus derived from M-MuLV), or or the double copy vectors designated DCT5A, DCT5B and DCT5C (see PCT Application US 90/02656).

Sense or antisense oligonucleotides also may be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell.

Alternatively, a sense or an antisense oligonucleotide may be introduced into a cell containing the target nucleic acid sequence by formation of an oligonucleotide-lipid complex, as described in WO 90/10448. The sense or antisense oligonucleotide-lipid complex is preferably dissociated within the cell by an endogenous lipase.

C. Flt3-Ligand Pharmaceutical Compositions

For in vivo administration to subjects and especially humans, Flt3-ligand can be formulated according to known methods used to prepare a Flt3-ligand composition, such as pharmaceutical compositions. Flt3-ligand can be combined in admixture, either as the sole active material or with other known active materials, with pharmaceutically suitable diluents (e.g., Tris-HCl, acetate, phosphate), preservatives (e.g., Thimerosal, benzyl alcohol, parabens), emulsifiers, solubilizers, adjuvants and/or carriers. The term pharmaceutically acceptable means a non-toxic material that does not interfere with the effectiveness of the biological activity of the active ingredient(s). Suitable carriers and their formulations are described in Remington's Pharmaceutical Sciences, 16th ed. 1980, Mack Publishing Co. In addition, such compositions can contain Flt3-ligand complexed with polyethylene glycol (PEG)- or other such compounds to increase solubility and/or pharmacokinetic half-life, metal ions, or incorporated into polymeric compounds such as polyacetic acid, polyglycolic acid, hydrogels, etc., or incorporated into liposomes, microemulsions, micelles, unilamellar or multilamellar vesicles, erythrocyte ghosts or spheroblasts. Such compositions will influence the physical state, solubility, stability, rate of in vivo release, and rate of in vivo clearance of Flt3-ligand.

Flt3-ligand pharmaceutical compositions can be administered topically, parenterally, or by inhalation. The term “parenteral” includes subcutaneous injections, intravenous, intramuscular, intracisternal injection, or infusion techniques. These compositions will typically contain an effective amount of the Flt3-ligand, alone or in combination with an effective amount of any other active material. Such dosages and desired drug concentrations contained in the compositions may vary depending upon many factors, including the intended use, subject's body weight and age, and route of administration. Preliminary doses can be determined according to animal tests, and the scaling of dosages for human administration can be performed according to art-accepted practices. Keeping the above description in mind, typical dosages of Flt3-ligand may range from about 10 μg per square meter to about 1000 μg per square meter. A preferred dose range is on the order of about 100 μg per square meter to about 300 μg per square meter.

In practicing transplatation procedures incorporating administration of Flt3-ligand, a therapeutically effective amount of Flt3-ligand, and optionally an auxiliary molecule such as a growth factor are administered to a subject. As used herein, the term “effective amount” means the total amount of each therapeutic agent (i.e., Flt3-ligand, and optionally an auxiliary molecule) or other active component that is sufficient to show a meaningful benefit to the subject, i.e., enhanced immune response, treatment, healing, prevention or amelioration of the relevant medical condition (disease, infection, etc.), or an increase in rate of treatment, healing, prevention or amelioration of such conditions. When “effective amount” is applied to an individual therapeutic agent administered alone, the term refers to that therapeutic agent alone. When applied to a combination, the term refers to combined amounts of the ingredients that result in the therapeutic effect, whether administered in combination, serially or simultaneously. As used herein, the phrase “administering an effective amount” of a therapeutic agent means that the subject is treated with said therapeutic agent(s) in an amount and for a time sufficient to induce an improvement, and preferably a sustained improvement, in at least one indicator that reflects the severity of the disorder. An improvement is considered “sustained” if the patient exhibits the improvement on at least two occasions separated by one or more days, or one or more weeks. The degree of improvement is determined based on signs or symptoms, and determinations can also employ questionnaires that are administered to the patient, such as quality-of-life questionnaires. Various indicators that reflect the extent of the patient's illness can be assessed for determining whether the amount and time of the treatment is sufficient. The baseline value for the chosen indicator or indicators is established by examination of the patient prior to administration of the first dose of the therapeutic agent(s). Preferably, the baseline examination is done within about 60 days of administering the first dose. If the therapeutic agent(s) is/are being administered to treat acute symptoms, the first dose is administered as soon as practically possible. Improvement is induced by administering therapeutic agents until the subject manifests an improvement over baseline for the chosen indicator or indicators. In treating chronic conditions, this degree of improvement is obtained by repeatedly administering the therapeutic agents over a period of at least a month or more, e.g., for one, two, or three months or longer, or indefinitely. A period of one to six weeks, or even a single dose, may be sufficient for treating certain conditions. One of skill in the art would particularize the treatment to suit the subjects needs. Although the extent of the subject's illness after treatment may appear improved according to one or more indicators, treatment may be continued indefinitely at the same level or at a reduced dose or frequency. Once treatment has been reduced or discontinued, it later may be resumed at the original level if symptoms should reappear.

One skilled in the pertinent art will recognize that suitable dosages will vary, depending upon such factors as the nature and severity of the disorder to be treated, the patient's body weight, age, general condition, and prior illnesses and/or treatments, and the route of administration. Preliminary doses can be determined according to animal tests, and the scaling of dosages for human administration is performed according to art-accepted practices such as standard dosing trials. For example, the therapeutically effective dose can be estimated initially from cell culture assays. The dosage will depend on the specific activity of the compound and can be readily determined by routine experimentation. A dose can be formulated in animal models to achieve a circulating plasma concentration range that includes the IC50 (i.e., the concentration of the test compound which achieves a half-maximal inhibition of symptoms) as determined in cell culture, while minimizing toxicities. Such information can be used to more accurately determine useful doses in humans. Ultimately, the attending physician will decide the amount of polypeptide of the present invention with which to treat each individual patient. Initially, the attending physician will administer low doses of polypeptide of the present invention and observe the patient's response. Larger doses of polypeptide of the present invention can be administered until the optimal therapeutic effect is obtained for the patient, and at that point the dosage is not increased further. It is contemplated that the therapeutic agents used to practice the methods described herein should contain about 0.01 ng to about 100 mg (alternative embodiments have about 0.1 ng to about 10 mg, and other embodiments have about 0.1 microgram to about 1 mg) of polypeptide of the present invention per kg body weight. If a route of administration other than injection is used, the dose is appropriately adjusted in accord with standard medical practices. For incurable chronic conditions, the regimen can be continued indefinitely, with adjustments being made to dose and frequency if such are deemed necessary by the patient's physician.

Pharmaceutical compositions may also comprise Flt3-ligand combined with one or more auxiliary molecules, such as one or more growth factors, as well as a pharmaceutically acceptable diluent, carrier, or excipient, are encompassed by the invention. Alternatively, the auxiliary molecules may be formulated as a separate pharmaceutical composition. Formulations suitable for administration include aqueous and non-aqueous sterile injection solutions which can contain anti-oxidants, buffers, bacteriostats and solutes which render the formulation isotonic with the blood of the recipient; and aqueous and non-aqueous sterile suspensions which can include suspending agents or thickening agents. Flt3-ligand and/or auxiliary molecules can be formulated according to known methods used to prepare pharmaceutically useful compositions. They can be combined in admixture, either as the sole active material or with other known active materials suitable for a given indication, with pharmaceutically acceptable diluents (e.g., saline, Tris-HCl, acetate, and phosphate buffered solutions), preservatives (e.g., thimerosal, benzyl alcohol, parabens), emulsifiers, solubilizers, adjuvants and/or carriers. Suitable formulations for pharmaceutical compositions include those described in Remington's Pharmaceutical Sciences, 16th ed. 1980, Mack Publishing Company, Easton, Pa. In addition, such compositions can be complexed with polyethylene glycol (PEG), metal ions, or incorporated into polymeric compounds such as polyacetic acid, polyglycolic acid, hydrogels, dextran, etc., or incorporated into liposomes, microemulsions, micelles, unilamellar or multilamellar vesicles, erythrocyte ghosts or spheroblasts. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. No. 4,235,871; U.S. Pat. No. 4,501,728; U.S. Pat. No. 4,837,028; and U.S. Pat. No. 4,737,323. Such compositions will influence the physical state, solubility, stability, rate of in vivo release, and rate of in vivo clearance, and are thus chosen according to the intended application, so that the characteristics of the carrier will depend on the selected route of administration.

In one embodiment, sustained-release forms of Flt3-ligand and auxiliary molecules are used. Sustained-release forms suitable for use in the disclosed methods include, but are not limited to, Flt3-ligand and auxiliary molecules that are encapsulated in a slowly-dissolving biocompatible polymer (such as the alginate microparticles described in U.S. Pat. No. 6,036,978), admixed with such a polymer (including topically applied hydrogels), and or encased in a biocompatible semi-permeable implant.

One type of sustained release technology that may be used in administering soluble Flt3-L therapeutic compositions is that utilizing hydrogel materials, for example, photopolymerizable hydrogels (Sawhney et al., Macromolecules 26:581; 1993). Similar hydrogels have been used to prevent postsurgical adhesion formation (Hill-West et al., Obstet. Gynecol. 83:59, 1994) and to prevent thrombosis and vessel narrowing following vascular injury (Hill-West et al., Proc. Natl. Acad. Sci. USA 91:5967, 1994). Polypeptides can be incorporated into such hydrogels to provide sustained, localized release of active agents (West and Hubbel, Reactive Polymers 25:139, 1995; Hill-West et al., J. Surg. Res. 58:759; 1995). The sustained, localized release Flt3-L when incorporated into hydrogels would be amplified by the long half life of Flt3-L.

The compounds of this invention can be included in the formulation as fine multiparticulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the protein (or derivative) may be formulated (such as by liposome or microsphere encapsulation) and then further contained within an edible product, such as a refrigerated beverage containing colorants and flavoring agents.

One may dilute or increase the volume of the compound of the invention with an inert material. These diluents could include carbohydrates, especially mannitol, α-lactose, anhydrous lactose, cellulose, sucrose, modified dextrans and starch. Certain inorganic salts may also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrants include but are not limited to starch including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An antifrictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the compound of this invention into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethonium chloride. The list of potential nonionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the protein or derivative either alone or as a mixture in different ratios.

Additives may also be included in the formulation to enhance uptake of the compound. Additives potentially having this property are for instance the fatty acids oleic acid, linoleic acid and linolenic acid.

Controlled release formulation may be desirable. The compound of this invention could be incorporated into an inert matrix which permits release by either diffusion or leaching mechanisms; e.g., gums. Slowly degenerating matrices may also be incorporated into the formulation, e.g., alginates, polysaccharides. Another form of a controlled release of the compounds of this invention is by a method based on the Oros therapeutic system (Alza Corp.), i.e., the drug is enclosed in a semipermeable membrane which allows water to enter and push drug out through a single small opening due to osmotic effects. Some enteric coatings also have a delayed release effect.

Other coatings may be used for the formulation. These include a variety of sugars which could be applied in a coating pan. The therapeutic agent could also be given in a film coated tablet and the materials used in this instance are divided into 2 groups. The first are the nonenteric materials and include methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone and the polyethylene glycols. The second group consists of the enteric materials that are commonly esters of phthalic acid.

A mix of materials might be used to provide the optimum film coating. Film coating may be carried out in a pan coater or in a fluidized bed or by compression coating.

Also contemplated herein is pulmonary delivery of the present protein (or derivatives thereof). The protein (or derivative) is delivered to the lungs of a mammal while inhaling and traverses across the lung epithelial lining to the blood stream. (Other reports of this include Adjei et al., Pharma. Res. (1990) 7: 565-9; Adjei et al. (1990), Internatl. J. Pharmaceutics 63: 135-44 (leuprolide acetate); Braquet et al. (1989), J. Cardiovasc. Pharmacol. 13 (suppl.5): s.143-146 (endothelin-1); Hubbard et al. (1989), Annals Int. Med. 3: 206-12 (α1-antitrypsin); Smith et al. (1989), J. Clin. Invest. 84: 1145-6 (α1-proteinase); Oswein et al. (March 1990), “Aerosolization of Proteins”, Proc. Symp. Resp. Drug Delivery II, Keystone, Colorado (recombinant human growth hormone); Debs et al. (1988), J. Immunol. 140: 3482-8 (interferon-γ and tumor necrosis factor α) and Platz et al., U.S. Pat. No. 5,284,656 (granulocyte colony stimulating factor).

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art. Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colorado; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of the inventive compound. Typically, each formulation is specific to the type of device employed and may involve the use of an appropriate propellant material, in addition to diluents, adjuvants and/or carriers useful in therapy.

The inventive compound should most advantageously be prepared in particulate form with an average particle size of less than 10 μm (or microns), most preferably 0.5 to 5 μm, for most effective delivery to the distal lung.

Pharmaceutically acceptable carriers include carbohydrates such as trehalose, mannitol, xylitol, sucrose, lactose, and sorbitol. Other ingredients for use in formulations may include DPPC, DOPE, DSPC and DOPC. Natural or synthetic surfactants may be used. PEG may be used (even apart from its use in derivatizing the protein or analog). Dextrans, such as cyclodextran, may be used. Bile salts and other related enhancers may be used. Cellulose and cellulose derivatives may be used. Amino acids may be used, such as use in a buffer formulation.

Also, the use of liposomes, microcapsules or microspheres, inclusion complexes, or other types of carriers is contemplated.

Formulations suitable for use with a nebulizer, either jet or ultrasonic, will typically comprise the inventive compound dissolved in water at a concentration of about 0.1 to 25 mg of biologically active protein per mL of solution. The formulation may also include a buffer and a simple sugar (e.g., for protein stabilization and regulation of osmotic pressure). The nebulizer formulation may also contain a surfactant, to reduce or prevent surface induced aggregation of the protein caused by atomization of the solution in forming the aerosol.

Formulations for use with a metered-dose inhaler device will generally comprise a finely divided powder containing the inventive compound suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing the inventive compound and may also include a bulking agent, such as lactose, sorbitol, sucrose, mannitol, trehalose, or xylitol in amounts which facilitate dispersal of the powder from the device, e.g., 50 to 90% by weight of the formulation. Alternative embodiments further include administration of Flt3-ligand concurrently with one or more other auxiliary molecules administered to the same subject. It is understood that the Flt3-ligand and auxiliary molecule(s) are administered as pharmaceutical compositions. Concurrent administration encompasses simultaneous or sequential treatment with Flt3-ligand and/or auxiliary molecules, as well as protocols in which the components are alternated, or wherein one component is administered long-term and the other(s) are administered intermittently. Components, i.e., Flt3-ligand and one or more auxiliary molecules, can be administered in the same or in separate compositions, and by the same or different routes of administration. Examples of auxiliary molecules that can be administered concurrently with Flt3-ligand include: cytokines, growth factors and the like will be useful in further enhancing or modulating an immune response. Cytokines include, but are not limited to those selected from the group comprising Interleukins 1, 2, 3, 4, 5, 6, 7, 10, 12, 15, 18 and 23, chemokines, GM-CSF, G-CSF, Interferon-alpha and gamma, c-kit ligand, fusions of GM-CSF and IL-3, TNF family members (TNF-α), TGF-β, soluble CD40 ligand, CD40-binding proteins, soluble CD83, 4-1BB binding proteins, OX-40 binding proteins, CpG sequences, and combinations thereof.

Routes of Administration. Any efficacious route of administration can be used to therapeutically administer Flt3-ligand, one or more auxiliary molecules and one or more vaccines. Parenteral administration includes injection, for example, via intra-articular, intravenous, intramuscular, intralesional, intraperitoneal or subcutaneous routes by bolus injection or by continuous infusion., and also includes localized administration, e.g., at a site of disease or injury. Other suitable means of administration include sustained release from implants; aerosol inhalation and/or insufflation; eyedrops; vaginal or rectal suppositories; buccal preparations; oral preparations, including pills, syrups, lozenges, ice creams, or chewing gum; and topical preparations such as lotions, gels, sprays, ointments or other suitable techniques. Cells may also be cultured ex vivo in the presence of Flt3-ligand, one or more auxiliary molecules and one or more vaccines in order to modulate cell proliferation or to produce a desired effect on or activity in such cells. Treated cells can then be introduced in vivo for therapeutic purposes. When Flt3-ligand, one or more auxiliary molecules and one or more vaccines are administered to a subject, these can be administered by the same or by different routes, and can be administered simultaneously, separately or sequentially.

Oral Administration. When a therapeutically effective amount of Flt3-ligand, one or more auxiliary molecules and one or more vaccines are administered orally, they may be in the form of a tablet, capsule, powder, solution or elixir. When administered in tablet form, the Flt3-ligand, one or more auxiliary molecules and one or more vaccines can additionally contain a solid carrier such as a gelatin or an adjuvant. The tablet, capsule, and powder contain from about 5 to 95% polypeptide of the present invention, and preferably from about 25 to 90% polypeptide of the present invention. Contemplated for use herein are oral solid dosage forms, which are described generally in Chapter 89 of Remington's Pharmaceutical Sciences (1990), 18th Ed., Mack Publishing Co. Easton Pa. 18042, which is herein incorporated by reference in its entirety. Solid dosage forms include tablets, capsules, pills, troches or lozenges, cachets or pellets. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). A description of possible solid dosage forms for the therapeutic is given in Chapter 10 of Marshall, K., Modern Pharmaceutics (1979), edited by G. S. Banker and C. T. Rhodes, herein incorporated by reference in its entirety. In general, the formulation will include Flt3-L and inert ingredients which allow for protection against the stomach environment, and release of the biologically active material in the intestine.

Also specifically contemplated are oral dosage forms of the above, inventive compounds. If necessary, the compounds may be chemically modified so that oral delivery is efficacious. Generally, the chemical modification contemplated is the attachment of at least one moiety to the compound molecule itself, where said moiety permits (a) inhibition of proteolysis; and (b) uptake into the blood stream from the stomach or intestine. Also desired is the increase in overall stability of the compound and increase in circulation time in the body. Moieties useful as covalently attached vehicles in this invention may also be used for this purpose. Examples of such moieties include: PEG, copolymers of ethylene glycol and propylene glycol, carboxymethyl cellulose, dextran, polyvinyl alcohol, polyvinyl pyrrolidone and polyproline. See, for example, Abuchowski and Davis, Soluble Polymer-Enzyme Adducts, Enzymes as Drugs (1981), Hocenberg and Roberts, eds., Wiley-Interscience, New York, N.Y., pp. 367-83; Newmark, et al. (1982), J. Appl. Biochem. 4:185-9. Other polymers that could be used are poly-1,3-dioxolane and poly-1,3,6-tioxocane. Preferred for pharmaceutical usage, as indicated above, are PEG moieties. For oral delivery dosage forms, it is also possible to use a salt of a modified aliphatic amino acid, such as sodium N-(8-[2-hydroxybenzoyl]amino) caprylate (SNAC), as a carrier to enhance absorption of the therapeutic compounds of this invention. The clinical efficacy of a heparin formulation using SNAC has been demonstrated in a Phase II trial conducted by Emisphere Technologies. See U.S. Pat. No. 5,792,451, “Oral drug delivery composition and methods”.

When administered in liquid form, a liquid carrier such as water, petroleum, oils of animal or plant origin such as peanut oil, mineral oil, soybean oil, or sesame oil, or synthetic oils can be added. The liquid form of Flt3-ligand, one or more auxiliary molecules and one or more vaccines can further contain physiological saline solution, dextrose or other saccharide solution, or glycols such as ethylene glycol, propylene glycol or polyethylene glycol.

Intravenous Administration. When a therapeutically effective amount of Flt3-ligand, one or more auxiliary molecules and one or more vaccines is administered by intravenous, cutaneous or subcutaneous injection, Flt3-ligand, one or more auxiliary molecules and one or more vaccines may be in the form of a pyrogen-free, parenterally acceptable aqueous solution. The preparation of such parenterally acceptable polypeptide solutions, having due regard to pH, isotonicity, stability, and the like, is within the skill in the art. A preferred pharmaceutical composition for intravenous, cutaneous, or subcutaneous injection should contain, in addition to polypeptide of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical compositions of the present invention can also contain stabilizers, preservatives, buffers, antioxidants, or other additives known to those of skill in the art. The duration of intravenous therapy using the pharmaceutical compositions of the present invention will vary, depending on the severity of the disease being treated and the condition and potential idiosyncratic response of each individual patient. It is contemplated that the duration of each application of the polypeptide of the present invention will be in the range of 12 to 24 hours of continuous intravenous administration. Ultimately the attending physician will decide on the appropriate duration of intravenous therapy using the pharmaceutical compositions of the present invention.

Tissue Administration. Flt3-ligand, one or more auxiliary molecules and one or more vaccines of the present invention may be administered topically, systematically, or locally as an implant or device. When administered, the Flt3-ligand, one or more auxiliary molecules and one or more vaccines is, of course, in a pyrogen-free, physiologically acceptable form. Further, the Flt3-ligand, one or more auxiliary molecules and one or more vaccines can be encapsulated or injected in a viscous form for delivery to a desired site. Topical administration of Flt3-ligand, one or more auxiliary molecules and/or one or more vaccines is also envisioned for alternative embodiments of Flt3-ligand immunization protocols.

In addition to the above, the following examples are provided to illustrate particular embodiments and not to limit the scope of the invention.

### EXAMPLES

### Example 1

### Use of Flt3-L Alone and in Combination with IL-7 or IL-3

This example demonstrates the stimulation and proliferation of AA4.1⁺ fetal liver cells by compositions containing flt3-L and IL-7; as well as the stimulation and proliferation of c-kit-positive (c-kit⁺) cells by compositions containing flt3-L and IL-3.

AA4.1-positive (AA4.1⁺) expressing cells were isolated from the livers of day 14 fetal C57BL/6 mice by cell panning in Optilux 100 mm plastic Petri dishes (Falcon No. 1001, Oxnard, Calif.). Plates were coated overnight at 4° C. in PBS plus 0.1% fetal bovine serum (FBS) containing 10 μg/ml AA4.1 antibody (McKearn et. al., J. Immunol., 132:332-339, 1984) and then washed extensively with PBS plus 1% FBS prior to use. A single cell suspension of liver cells was added at 10⁷ cells/dish in PBS plus 1% FBS and allowed to adhere to the plates for two hours at 4° C. The plates were then extensively washed, and the adhering cells were harvested by scraping for analysis or further use in the hematopoiesis assays described below. FACS analysis using AA4.1 antibody demonstrated a >95% AA4.1⁺ cell population.

C-kit⁺ pluripotent stem cells were purified from adult mouse bone marrow (de Vries et. al., J. Exp. Med., 176:1503-1509, 1992; and Visser and de Vries, Methods in Cell Biol., 1993, submitted). Low density cells (≦1.078 g/cm³) positive for the lectin wheat germ agglutinin and negative for the antigens recognized by the B220 and 15-1.4.1 (Visser et. al., Meth. in Cell Biol., 33:451-468, 1990) monoclonal antibodies, could be divided into sub-populations of cells that do and do not express c-kit by using biotinylated Steel factor. The c-kit⁺ fraction has been shown to contain pluripotent hematopoietic stem cells (de Vries et. al., Science 255:989-991, 1992; Visser and de Vries, Methods in Cell Biol., 1993, submitted; and Ware et. al., 1993, submitted).

AA4.1+ Fetal liver cells were cultured in recombinant IL-7 (U.S. Pat. No. 4,965,195) at 100 ng/ml and recombinant flt3-L at 250 ng/ml. Flt3-L was used in three different forms in the experiments: (1) as present on fixed, flt3-L-transfected CV1/EBNA cells; (2) as concentrated culture supernatants from these same flt3-L-transfected CV1/EBNA cells; and (3) as a purified and isolated polypeptide preparation from yeast supernatant as described in Example 5.

Hematopoiesis Assays

The proliferation of c-kit⁺ stem cells, fetal liver AA4.1⁺ cells was assayed in [3H]-thymidine incorporation assays as essentially described by deVries et. al., J. Exp. Med., 173:1205-1211, 1991. Purified c-kit⁺ stem cells were cultured at 37° C. in a fully humidified atmosphere of 6.5% CO₂ and 7% O₂ in air for 96 hours. Murine recombinant IL-3 was used at a final concentration of 100 ng/ml. Subsequently, the cells were pulsed with 2 μCi per well of [³H]-thymidine (81 Ci/mmol; Amersham Corp., Arlington Heights, Ill.) and incubated for an additional 24 hours. AA4.1⁺ cells (approximately 20,000 cells/well) were incubated in IL-7, flt3-L and flt3-L+IL-7 for 48 hours, followed by [³H]-thymidine pulse of six hours. The results of flt3-L and IL-7 are shown in Table I, and results of flt3-L and IL-3 are shown in Table II.

TABLE I
Effect of Flt3-L and IL-7 on Proliferation of AA4.1 + Fetal Liver Cells.

| | | Factor | | |
|---|---|---|---|---|
| | Control | flt3-L | IL-7 | flt3-L + IL-7 |
| [³H]-thymidine | 100 | 1000 | 100 | 4200 |
| incorporation | | | | |

(CPM)

The combination of flt3-L and IL-7 produced a response that was approximately four-fold greater than flt3-L alone and approximately 40-fold greater than IL-7 alone.

TABLE II
Effect of Flt3-L and IL-3 on Proliferation of C-kit + Cells.

| | | Factor | | |
|---|---|---|---|---|
| | Control (vector alone) | flt3-L | IL-3 | flt3-L + IL-3 |
| [³H]-thymidine | 100 | 1800 | 3000 | 9100 |
| incorporation | | | | |
| (CPM) | | | | |

Culture supernatant from CV1/EBNA cells transfected with flt3-L cDNA stimulated the proliferation of c-kit⁺ stem cells approximately 18-fold greater than the culture supernatant of CV1/EBNA cells transfected with the expression vector alone. Addition of IL-3 to flt3-L containing supernatant showed a synergistic effect, with approximately twice the degree of proliferation observed than would be expected if the effects were additive.

### Example 2

### Flt3-L Stimulates Proliferation of Erythroid Cells in the Spleen

This example describes the effect of flt3-L on the production of erythroid cells in the spleen of transgenic mice. Transgenic mice were generated according to the procedures of Example 9. The mice were sacrificed and each intact spleen was made into a single cell suspension. The suspended cells were spun and then resuspended in 10 ml of medium that contained PBS+1% fetal bovine serum. Cell counts were performed thereon using a hemocytometer. Each cell specimen was counted with Trypan Blue stain to obtain a total viable cell count per milliliter of medium according to the following formula: (RBC+WBC)/ml, wherein RBC is the red blood cell count and WBC means the white blood cell count. Each specimen then was counted with Turk's stain to obtain a total white blood cell count per milliliter of medium. The total red blood cell count per milliliter was calculated for each specimen by subtracting the total white blood cell count per milliliter from the total viable cell count per milliliter. The results are shown in the following Table III.

TABLE III
Erythroid Cell Proliferation in Flt3-L-Overexpressing
Transgenic Mice Spleen

| | | | Total |
|---|---|---|---|
| | Total Viable Cell | Total White Cell | Red Blood Cell |
| Mouse | (million cells/ml) | (million cells/ml) | (million cells/ml) |
| Control 1 | 29.7 | 27 | 2.7 |
| Control 2 | 31 | 24.6 | 6.4 |
| Transgenic 1 | 44.7 | 25.6 | 19.1 |
| Transgenic 2 | 37.3 | 28.4 | 8.9 |

From the data of Table III, the white blood cell counts per milliliter were approximately the same as the control mice. However, the red blood cell counts from the spleens of the two transgenic mice were approximately two to three-fold greater than observed in the control mice. Flt3-L stimulates an increase in cells of the erythroid lineage, possibly through stimulation of erythroid proogenitor cells, through the stimulation of cells that produce erythropoietin, or by blocking a mechanism that inhibits erythropoiesis.

### Example 3

### Flt3-L Stimulates Proliferation of T Cells and Early B Cells

Bone marrow from 9 week old transgenic mice generated according to Example 9 was screened for the presence of various T and B cell phenotype markers using antibodies that are immunoreactive with such markers. The following markers were investigated: the B220 marker, which is specific to the B cell lineage; surface IgM marker (sIgM), which is specific to mature B cells; the S7 (CD43) marker, which is an early B cell marker; the Stem Cell Antigen-1 (SCA-1) marker, which is a marker of activated T cells and B cells; CD4, which is a marker for helper T cells and some stem cells; and the Mac-1 marker, which is specific to macrophages, were screened using well known antibodies against such markers. The following Table IV shows the data obtained from screening the bone marrow. Two transgenic mice from the same litter were analyzed against a normal mouse from the same litter (control), and an unrelated normal mouse (control).

TABLE IV
Effect of flt3-L Overexpression in Transgenic Mice

Percentage of Positive Cells

| | Unrelated | Littermate | | |
|---|---|---|---|---|
| Marker | Control | Control | Transgenic #1 | Transgenic #2 |
| B220 | 30.64 | 27.17 | 45.84 | 48.78 |
| sIgM | 3.54 | 2.41 | 1.94 | 1.14 |
| S7(CD43) | 54.43 | 45.44 | 46.11 | 50.59 |
| SCA-1 | 10.92 | 11.74 | 19.45 | 27.37 |
| CD4 | 6.94 | 8.72 | 12.21 | 14.05 |
| Mac-1 | 36.80 | 27.15 | 21.39 | 18.63 |

The above data indicate that flt3-L overexpression in mice leads to an increase in the number of B cells, as indicated by the increase B220⁺ cells and SCA-1⁺ cells. Analysis of B220⁺ cells by FACS indicated an increase in proB cells (HSA⁻, S7⁺). The increase in CD4⁺ cells indicated an approximate two-fold increase in T cells and stem cells. The decrease in cells having the sIgM marker indicated that flt3-L does not stimulate proliferation of mature B cells. These data indicate that flt3-L increases cells with a stem cell, T cell or an early B cell phenotype, and does not stimulate proliferation of mature B cells or macrophages.

### Example 4

### Use of Flt3-L in Peripheral Stem Cell Transplantation

This Example describes a method for using Flt3-L in autologous peripheral stem cell (PSC) or peripheral blood progenitor cell (PBPC) transplantation. Typically, PBPC and PSC transplantation is performed on patients whose bone marrow is unsuitable for collection due to, for example, marrow abnormality or malignant involvement.

Prior to cell collection, it may be desirable to mobilize or increase the numbers of circulating PBPC and PSC. Mobilization can improve PBPC and PSC collection, and is achievable through the intravenous administration of flt3-L to the patients prior to collection of such cells. Other growth factors such as CSF-1, GM-CSF, SF, G-CSF, EPO, IL-1, IL-2, IL-3, IL-4, IL-5, IL-6, IL-7, IL-8, IL-9, IL-10, IL-11, IL-12, IL-13, IL-14, IL-15, GM-CSF/IL-3 fusion proteins, LIF, FGF and combinations thereof, can be likewise administered in sequence, or in concurrent combination with flt3-L. Mobilized or non-mobilized PBPC and PSC are collected using apheresis procedures known in the art. See, for example, Bishop et al., Blood, vol. 83, No. 2, pp. 610-616 (1994). Briefly, PBPC and PSC are collected using conventional devices, for example, a Haemonetics Model V50 apheresis device (Haemonetics, Braintree, Mass.). Four-hour collections are performed typically no more than five times weekly until approximately 6.5×10⁸ mononuclear cells (MNC)/kg patient are collected. Aliquots of collected PBPC and PSC are assayed for granulocyte-macrophage colony-forming unit (CFU-GM) content by diluting approximately 1:6 with Hank's balanced salt solution without calcium or magnesium (HBSS) and layering over lymphocyte separation medium (Organon Teknika, Durham, N.C.). Following centrifugation, MNC at the interface are collected, washed and resuspended in HBSS. One milliliter aliquots containing approximately 300,000 MNC, modified McCoy's 5A medium, 0.3% agar, 200 U/mL recombinant human GM-CSF, 200 u/mL recombinant human IL-3, and 200 u/mL recombinant human G-CSF are cultured at 37° C. in 5% CO₂ in fully humidified air for 14 days. Optionally, flt3-L or GM-CSF/IL-3 fusion molecules (PIXY 321) may be added to the cultures. These cultures are stained with Wright's stain, and CFU-GM colonies are scored using a dissecting microscope (Ward et al., Exp. Hematol., 16:358 (1988). Alternatively, CFU-GM colonies can be assayed using the CD34/CD33 flow cytometry method of Siena et al., Blood, Vol. 77, No. 2, pp 400-409 (1991), or any other method known in the art.

CFU-GM containing cultures are frozen in a controlled rate freezer (e.g., Cryo-Med, Mt. Clemens, Mich.), then stored in the vapor phase of liquid nitrogen. Ten percent dimethylsulfoxide can be used as a cryoprotectant. After all collections from the patient have been made, CFU-GM containing cultures are thawed and pooled. The thawed cell collection either is reinfused intravenoulsy to the patient or expanded ex vivo prior to reinfusion. Ex vivo expansion of pooled cells can be performed using flt3-L as a growth factor either alone, sequentially or in concurrent combination with other cytokines listed above. Methods of such ex vivo expansion are well known in the art. The cells, either expanded or unexpanded, are reinfused intravenously to the patient. To facilitate engraftment of the transplanted cells, flt3-L is administered simultaneously with, or subsequent to, the reinfusion. Such administration of flt3-L is made alone, sequentially or in concurrent combination with other cytokines selected from the list above.

### Example 5

### Purification of Hematopoietic Progenitor and Stem Cells Using Flt3-L

This Example describes a method for purifying hematopoietic progenitor cells and stem cells from a suspension containing a mixture of cells. Cells from bone marrow and peripheral blood are collected using conventional procedures. The cells are suspended in standard media and then centrifuged to remove red blood cells and neutrophils. Cells located at the interface between the two phases (also known in the art as the buffy coat) are withdrawn and resuspended. These cells are predominantly mononuclear and represent a substantial portion of the early hematopoietic progenitor and stem cells. The resulting cell suspension then is incubated with biotinylated Flt3-L for a sufficient time to allow substantial Flt3:Flt3-L interaction. Typically, incubation times of at least one hour are sufficient. After incubation, the cell suspension is passed, under the force of gravity, through a column packed with avidin-coated beads. Such columns are well known in the art, see Berenson, et al., J. Cell Biochem., 10D:239 (1986). The column is washed with a PBS solution to remove unbound material. Target cells can be released from the beads and from flt3-L using conventional methods.

### Example 6

### Flt3 Ligand Promotes Engraftment of Allogeneic Hematopoietic Stem and Progenitor Cells Using a Nonmyeloablative Conditioning Regimen

Experimental Animals

Litters of random-bred dogs were raised at the Fred Hutchinson Cancer Research Center (FHCRC—Seattle, Wash.) or obtained from commercial kennels licensed by the U.S. Department of Agriculture. The dogs weighed a median of 9 kg (range, 7-15.3 kg) and were a median of 10 (range, 7-30) months old. Dogs were observed for indications of disease for at least 60 days before entry into the transplant regimen. All were immunized against leptospirosis, distemper, hepatitis, parvovirus, and papillomavirus. Research was performed according to the principles outlined in the Guide for the Care and Use of Laboratory Animals, Institute of Laboratory Animal Resources, National Research Council. All dogs were housed in kennels certified by the American Association for Accreditation of Laboratory Animal Care. All dogs were examined at least twice daily. The research protocols were approved by the Institutional Animal Care and Use Committee of the FHCRC.

DLA Typing

Littermates were identified as DLA-identical donor-recipient on the basis of studies of highly polymorphic major histocompatibility complex (MHC) class I and class II microsatellite markers (Wagner J L, et al., Transplantation 1996; 62: 876). Specific DLA-DRB1 allelic identity was determined by direct sequencing.

Flt3-L Administration

The recombinant human Flt3-L protein was supplied as a sterile lyophilized preparation of 1.5 mg of Flt3-L, with 40 mg mannitol, 10 mg sucrose, and 25 mM of trimethamine per vial. Part of the stock was provided dissolved in 25 mM sodium phosphate solution at pH 7.2 and at 9.72 mg/mL concentration. Flt3-L was reconstituted before administration in sterile water for injection. Dose and schedule of administration of Flt3-L were translated and modified from phase I dose-escalation studies of Flt3-L administration to normal healthy human volunteers (Maraskovsky E, et al., Blood 2000; 96: 878).

Flt3-L was given subcutaneously at a dosage of 100 μg/kg once daily for 13 days to three healthy nonirradiated dogs. Three more normal dogs were used for the daily blood count monitoring for 14 days as a control group. Nine pairs of DLA-identical littermates were used in a DLA-identical model of allogeneic BM transplantation. Recipient dogs received FL subcutaneously at a dosage of 100 μg/kg once daily from day −7 to day +5.

TBI and Marrow Transplantation

Bone Marrow (BM) recipients were given TBI at a dose 4.5 Gy delivered at 0.07 Gy/min from a Clinac 4 linear accelerator (JM Company, San Jose, Calif.). In a preliminary dosimetry experiment with a dog-imitating phantom, appropriate instrument settings, distance from the beam source, and orientation and positioning of the dogs were established to ensure homogeneous delivery of the radiation dose to the point halfway though the dog body at 0.07 Gy/min. Each dog was sedated with a combination of Torbugesic (0.3 mg/kg; Animal Health, Fort Dodge, Iowa) and acepromazine (1.7 mg/9 kg; Boehringer Ingelheim Vetmedica, Inc., St. Joseph, Mo.) and placed in a prone position into a plexiglas cage with height inserts added to restrict movements. The cage was then mounted onto a table, secured with ratchet straps, and placed 256 cm from origination of the beam and perpendicular to its direction. Proper orientation of sagittal and midtransverse planes of the irradiated dog was determined by the intersection of the laser beams from two sources placed at right angles to one another (Diamond NW Medical Physics, Overlake, Wash.). Total dosage of received radiation was determined by external in vivo dosimeter (Sun Nuclear Corp., Melbourne, Fla.) and given in equal halves to each side of the dog's body. Donor marrow was collected and infused intravenously within 4 hr of TBI as described previously.

DC Immunohistochemistry

Popliteal lymph nodes were harvested before and after Flt3-L administration to three healthy single dogs. Procedures were performed under general anesthesia with intravenous injections of fentanyl at a dose of 0.4 mg/kg ketamine hydrochloride (Innovar-Vet; Pitman-Moore, Inc., Washington Crossing, N.J.) at a dose of 42 mg/9 kg, and acepromazine at a dose of 1.7 mg/9 kg (Boehringer Ingelheim Vetmedica, Inc., St. Joseph, Mo.). Popliteal lymph nodes specimens were embedded in OCT compound (Miles Inc., Elkhart, Ind.) and snap-frozen in isopentane (2-methylbutane) cooled to the point of freezing in liquid nitrogen. Molds were stored at −70° C. before processing.

Murine monoclonal antibodies specific for canine CD1c (CA13.9H11), CD11b (CA16.3E10), CD11c (CA11.6A1), CD11d (CA11.8H2), and MHC class II (CA2.1C12) were used for frozen section immunohistochemistry. A murine monoclonal antibody specific for feline CD1a (FE1.5F4) was used as a negative control.

Cryosections were fixed in acetone (3 min), and endogenous peroxidase was quenched by immersing slides in hydrogen peroxide (0.3%), sodium azide (0.1%), and phosphate-buffered saline (PBS) for 10 min. After a blocking step with 10% heat-inactivated horse serum in PBS (20 min), the canine-specific monoclonal antibodies were applied to the sections for 30 min. Appropriate antibody dilutions, prepared in 10% horse serum in PBS, were determined by previous titration of each antibody on frozen sections of normal canine spleen. Application of an isotype-matched nonspecific antibody was used as a negative control in each run. Secondary biotinylated horse anti-mouse immunoglobulin G (Vector, Burlingame, Calif.) was applied on each section for 30 min followed by streptavidin-horseradish peroxidase (Zymed, South San Francisco, Calif.) for 20 min according to instructions of the manufacturers. Between each step, the sections were washed thoroughly in PBS. Amino-9-ethyl-carbazole (AEC; Sigma Chemical Co., St. Louis, Mo.) was used as the chromogen. Finally, the tissue sections were counterstained with hematoxylin (Gill's formula No. 3; Fisher Scientific, Fairlawn, N.J.), air-dried, and cover-slipped.

Flow Cytometry

Peripheral blood specimens were collected in heparin tubes before and after 7 days of administration of Flt3-L and processed as described. Monoclonal antibodies were titrated to yield optimal immunofluorescence. Combination of CD45 Biotin (CA 12.10C12) used with streptavidin allophycocyanin (Caltag, Burlingame, Calif.), and anti-human CD14 conjugated with phycoerythrin (Tük4; DAKO, Carpinteria, Calif.) was used to enumerate lymphoid, monocyte, and myeloid cell populations of peripheral blood with acquisition of 10,000 events. The list mode data were analyzed using WinList software (Verity Software House, Topsham, Me.).

Mixed Lymphocyte Reaction

Peripheral blood mononuclear cells (PBMC) from Flt3-L-treated dogs were examined for their capacity to stimulate proliferation of alloreactive T cells from DLA-mismatched unrelated dogs in one-way mixed leukocyte reactions (MLR). Total PBMC fractions from the recipient dogs were isolated on Ficoll-Hypaque gradient before and at the end of the FL course, cryopreserved, and subsequently tested as stimulators against PBMC from DLA-mismatched unrelated dog and autologous PBMC. Proliferative responses were measured by standard MLR assays using ³H-thymidine uptake. Four, 5, and 6 days after triplicate plating of 1×10⁵ responding PBMC per well, cells were pulsed with 0.037 MBq per well of tritium thymidine and harvested after 18 hr. Counts per minute (CPM) were measured with a [beta]-scintillation counter (Packard, Meriden, Conn.), and results were presented as mean CPM±SEM or, for MLR, as the stimulation index (mean experimental CPM divided by mean autologous control CPM).

Assessment of Engraftment and Chimerism

Hematopoietic engraftment was determined by means of sustained recoveries of granulocyte and platelet counts after the postirradiation nadirs and by documentation of donor-derived (CA)ₙ repeat polymorphism from peripheral blood cells and BM aspirates (Yu, C, et al., Transplantation 1994; 58: 701). Peripheral blood was obtained every week for 6 months and then every 2 weeks until the end of the study. Genomic DNA was extracted from the whole blood and from granulocyte and mononuclear cell fractions separated on a Ficoll-Hypaque gradient. Polymerase chain reaction (PCR)-based assay was performed using primers specific for informative microsatellite markers. The PCR conditions have been described previously (Yu, C, et al., supra). Mixed hematopoietic chimerism was quantified by estimating the proportion of donor-specific to host-specific DNA with use of the storage phosphorimaging technique (Molecular Dynamics, Sunnyvale, Calif.).

Skin Grafts

Procedures were performed under general inhalation anesthesia with isoflurane. Full-thickness skin grafts (10-12 cm²) were placed into flank areas and fixed with layered compressive dressings and interrupted sutures. Autologous, BM donor, and third-party skin grafts from unrelated dogs were transplanted simultaneously. Grafts were irrigated daily by normal saline solution through wound dressings. First change of wound dressing was performed at day 7. Grafts were kept protected under Xeroform dressing (Tyco/Healthcare, Mansfield, Mass.) thereafter up to day 21. Skin grafts were evaluated daily or every other day, and any changes observed were documented by photography. Biopsy specimens were taken for histologic evaluation from unrelated dog skin grafts at the time of clinically evident rejection and from autologous and BM donor skin grafts every 6 to 8 weeks. Specimens were fixed in 10% formalin and then embedded in paraffin. Cut sections were stained with hematoxylin-eosin and evaluated for presence and character of cellular infiltrates and distorted architecture.

Statistical Analysis

Comparisons of peripheral blood counts between control dogs and healthy dogs receiving FL were performed with a two-sample t test, after log transformation of the counts. In vitro proliferative responses against PBMC from FL-treated dogs were evaluated using paired t test. Engraftment rates in the FL-treated group after marrow transplantation were compared with a historical control group of 37 dogs that received a minimum of 2.0×10⁸ total nucleated cells (TNC) per kilogram in the graft. Historical control dogs were used because of the robust nature of the model over time. All control dogs were conditioned with a TBI dose of 4.5 Gy at a rate of 0.07 Gy/min, received a marrow graft from a DLA-identical littermate, and did not receive posttransplant immunosuppression. Unadjusted comparisons of engraftment rates of the FL-treated and the control dogs were made with a chi-square test. Median marrow cell doses of the FL-treated and control dogs were compared with a Mann-Whitney U test. Comparisons of engraftment rates in the two groups, adjusted for the cell dose, were performed using logistic regression analysis.

1. Effects of Flt3-L in Healthy Dogs

A. Flt3-L Increased Neutrophil and Monocyte Counts in Peripheral Blood.

No adverse reactions were observed in the three recipients of Flt3-L at a dosage of 100 μg/kg/day. There was an increase in the white blood cell (1.8-fold, P=0.02), neutrophil (1.8-fold, P=0.007), and monocyte (4-fold, P=0.02) counts of the peripheral blood at day 13 as compared with a control group of normal dogs. The observed increase in lymphocyte counts and decrease in platelet counts was not significant (1.4-fold, P=0.5; and 1.4-fold, P=0.06, respectively).

Flow cytometry studies were also conducted on peripheral blood samples, which confirmed the marked increase in the number of CD14⁺ cells (monocytes) and neutrophils at 7 and 13 days after Flt3-L administration.

B. Flt3-L Increased the Content of CD1c⁺ Cells with DC Morphology in Peripheral Lymph Nodes.

Evaluation of frozen sections of popliteal lymph nodes harvested from three healthy dogs before Flt3-L administration for baseline studies revealed dispersed CD1c⁺ cells with dendritic morphology located in the paracortex; they occurred as single cells and small aggregates. Cells with DC morphology were revealed in the same location in serial sections stained with antibodies specific for CD1c and MHC class II. In popliteal lymph nodes taken from the same dogs after 13 days of Flt3-L administration, there was a marked increase in content of CD1c⁺ DC in the paracortex of lymph nodes; aggregates of DC cells extended into the medullary cords. Individual DC were rare; most of them were observed to be in small aggregates or confluent sheets.

C. Flt3-L Increased Stimulating Activity of PBMC Against Unrelated Responder Cells.

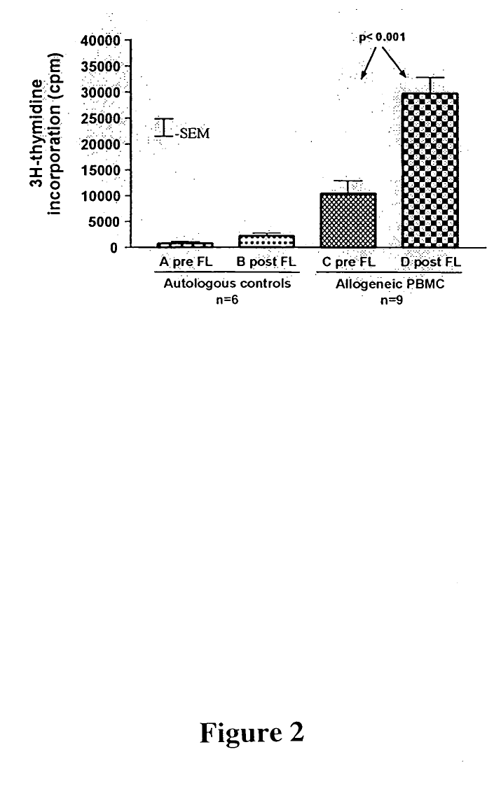

PBMC from Flt3-L-treated dogs (stimulators) harvested at day 13 of Flt3-L treatment enhanced proliferative response of unrelated PBMC (responders) at day 5 of MLC (10,438±2,442-29,761±3,091 counts/min) (P<0.001) (FIG. 2).

2. Effects of Flt3-L in Recipients of DLA-Identical Marrow

A. Flt3-L Promoted Engraftment of DLA-Identical Marrow.

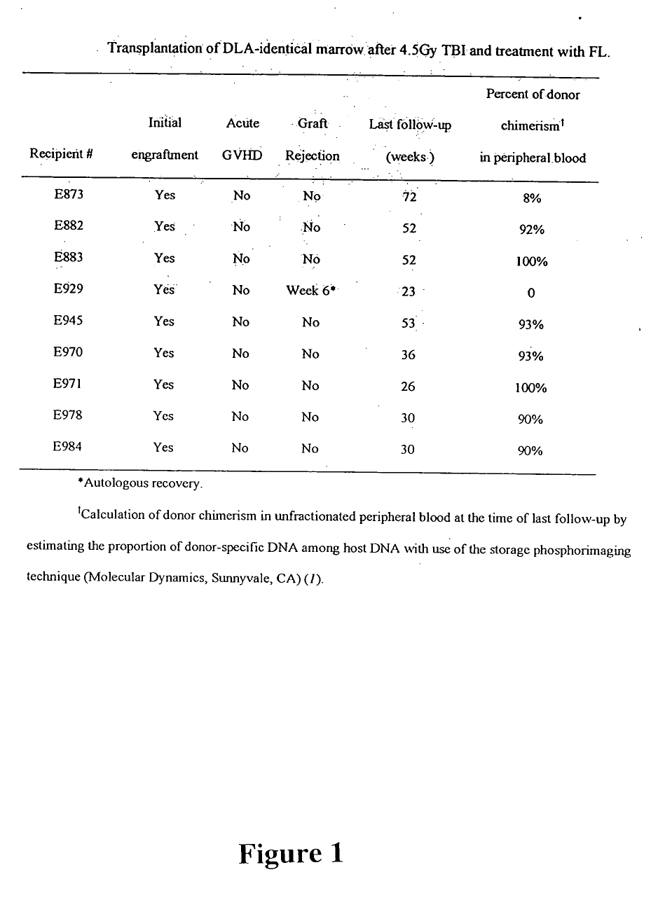

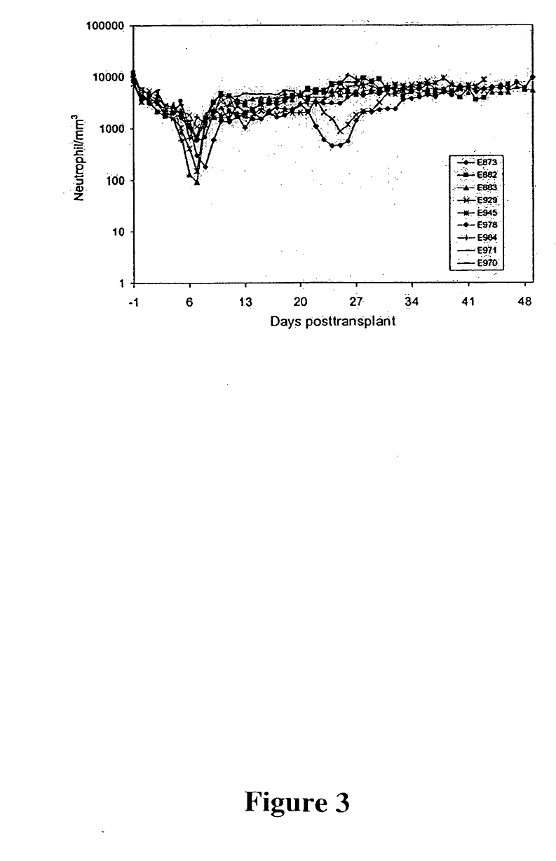

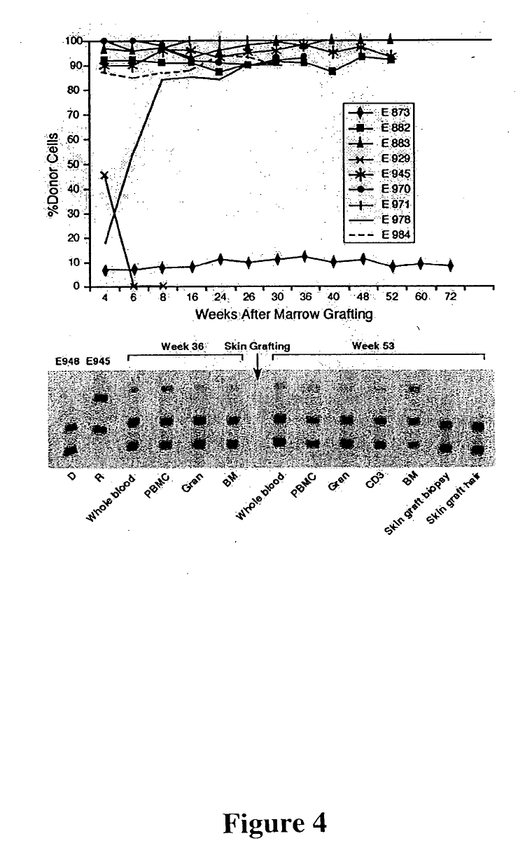

All nine recipients showed initial donor engraftment. Dogs had reached a median neutrophil count of 580 cells/4L (range, 91-1,020 cells/μL) at the nadir, 1 week after transplantation (FIG. 3). One dog (E929) had a second decrease in neutrophil count at week 4 after transplant and rejected the marrow graft between weeks 5 and 6, with subsequent autologous hematopoietic recovery. Another dog (E873) had a second neutrophil nadir and then recovery. This dog maintained a persistent low level of donor chimerism in unfractionated peripheral blood (8%), PBMC (7%), granulocytes (5%), marrow (12%), and CD3⁺ cells (16%) at last follow-up at 72 weeks posttransplant. All dogs are surviving with a median follow-up of 11 months (range, 7-18 months), and eight of the nine had sustained engraftment (FIG. 1). Two of the two engrafted dogs had complete donor chimerism in nucleated white blood cells and marrow. The median donor chimerism for the group was 93% (range, 8%-100%) (FIG. 4). The observed engraftment rate in the Flt3-L-treated group (eight of nine) compared with the control group (14 of 37) was significantly higher (P=0.004).

Despite a uniform, standardized procedure for marrow harvest, there was a difference in the median marrow cell doses between the Flt3-L-treatment and the control groups. The median TNC dose of donor marrow was 4.9×10⁸ cells/kg (range, 2.0-8.0×10⁸ cells/kg) for Flt3-L-treated dogs and 3.9×10⁸ cells/kg (range, 2.0-4.8×10⁸ cells/kg) for the control group (P=0.06). In a logistic regression analysis after adjusting for cell dose, Flt3-L administration was significantly associated with increased engraftment (P=0.02). None of the eight engrafted dogs in the Flt3-L-treated group developed graft-versus-host-disease (GVHD), compared with 3 of 14 in the control group.

B. Tolerance to Skin Grafts from Respective Marrow Donors is Established in Mixed Hematopoietic Chimeras.

Four of the eight engrafted dogs with sustained mixed hematopoietic chimerism were selected at random and had allogeneic skin grafts from both the respective marrow donors and unrelated DLA-mismatched dogs. Autologous skin grafts were transplanted simultaneously as a control. Grafts from unrelated DLA-mismatched donors were rejected within 7 to 9 days (median, 8 days). All autologous grafts and grafts from the marrow donors survived without loss. Before skin grafting, the donor hematopoietic chimerism levels on the four dogs were stable at 11%, 66%, 88%, and 94%. No changes in the level of donor hematopoietic chimerism were observed after skin grafting in any of the dogs.

In dogs, the risk of graft rejection increases after transplantation of marrow from DLA-identical littermates when the dose of TBI is reduced from 9.2 Gy and no posttransplant immunosuppression is administered. It had been previously reported that the engraftment rate dropped to 62% (15 of 24) and 34% (11 of 32) when the dose of TBI was reduced to 6 Gy and 4.5 Gy, respectively (Storb, R., et al., Blood 1994; 84: 3558). The addition of hematopoietic growth factors including granulocyte colony-stimulating factor (CSF) and stem cell factor, alone or in combination, did not significantly decrease the risk of graft rejection. In this transplant model using a reduced dose of TBI (4.5 Gy), graft rejection was observed in all dogs with the combination of interleukin (IL)-1-alpha and granulocyte-macrophage CSF after transplantation (n=4), prednisone after transplantation (n=5), or immunosuppressant cyclosporine (CSP) before transplantation (n=9) (Yu, C., et al., Blood 1995; 86:4376; Mathey, B., et al., Blood 1995; 86:833). The addition of viable donor peripheral blood mononuclear cells to the marrow graft at the time of transplant did not decrease the risk of graft rejection (Storb, R., et al., Transplantation 1995 59: 1481). However, the addition of a monoclonal antibody against the T cell (TCR-alpha-beta) before transplantation or cyclosporine (CSP) after marrow transplantation to suppress the host-versus-graft reaction resulted in five of the six and seven of the seven dogs achieving sustained engraftment, respectively (Barsoukov, A., et al., Transplantation 1999 67:1329). Thus, the dog model used in these studies is a robust model of allogeneic HSCT—as described in detail above, it was in this accepted dog model that Flt3-L was demonstrated to promote engraftment.

Flt3-L administration to mice has been shown to result in a marked increase in dendritic cells in both lymphoid and nonlymphoid tissues. When Flt3-L was administered to normal human volunteers, the number of functionally competent DC were expanded in the peripheral blood (Maraskovsky, E., et al., Blood 2000 96:878). In dogs, previous in vitro studies had shown that the human form of Flt3-L cross-reacted with canine cells, inducing DC from CD34-selected marrow cells (Hägglund, G., et al., Transplantation 2000 70:1437). In vivo, Flt3-L significantly increased the monocyte and neutrophil counts in the peripheral blood of dogs but slightly decreased platelets. Although an increase of DC in the peripheral blood of the dog could not be demonstrated because of the limited availability of reagents, there was an increase of DC in lymphoid tissue and an increase in the stimulatory capacity of the recipient's peripheral blood mononuclear cells in MLR. This confirmed that the effect of Flt3-L in dogs was similar to the reported effects of this cytokine in other species. The increased proliferative response of third-party responders to PBMC from Flt3-L-treated normal dogs in mixed lymphocyte culture (MLC) was consistent with the hypothesis that Flt3-L treatment of the recipient could increase alloimmune reactivity of the donor T cells to DC and hematopoietic cells of the DLA-identical recipients in vivo.

After allogeneic HSCT, recipient-derived antigen-presenting cells are necessary for inducing a GVH effect including GVHD (Shlomchik, W., et al., Science 1999; 285:412). Because DC are the most potent of the antigen-presenting cells, Flt3-L can be used to study the effect that DC have on transplant outcome. In mice, treatment of recipients with the combination of Flt3-L and tacrolimus without myelosuppression or other forms of immunosuppression enhances levels of donor hematopoietic cell chimerism more than either agent alone after transplantation of marrow (Antonysamy, M., et al., J Immunol 1998; 160:4106; Iyengar, A., et al. Transplantation 1997; 63:1193). In a murine model of solid-organ transplantation, the treatment of donors with Flt3-L before harvesting the organ resulted in augmentation of antidonor cytotoxic T-lymphocyte, natural-killer, and lymphokine-activated killer cell activities and enhanced rejection (Steptoe, R., et al., J Immunol 1997; 159:5483). Although studies on the effect of Flt3-L on alloimmune reactivity after transplantation are still limited in number, these observations in mice, in general, support the premise that depending on whether donor or recipient is treated, a GVH or host-versus-graft (HVG) reaction can be enhanced.

GVHD is one of the most significant complications that occurs after HSCT. However, a GVH reaction is important for the benefits that can be derived from allogeneic HSCT (Martin, P., et al., Blood 1998; 92:2177). The study described above showed tht Flt3-L could be administered safely to recipient dogs without the development of severe GVHD after allogeneic marrow transplantation from a DLA-identical littermate. Flt3-L treatment of recipients may have resulted in an increased GVH reaction that was predominantly directed against hematopoietic tissue without the development of severe GVHD. The observation that the GVH reaction can be predominantly directed against the hematopoietic system has been noted previously in mixed chimeric dogs infused with “sensitized” (recipient-specific) donor lymphocytes (Georges, G., et al., Blood 2000; 95:3262). The present study has not excluded an indirect effect of posttransplant Flt3-L on donor T cells, separate from the GVH reaction, which may have promoted engraftment. In contrast, lethal GVHD occurred in mice that had undergone transplant that had been treated with Flt3-L. The lack of significant GVHD in the dogs of the present study may have resulted from the dose and schedule of Flt3-L. In the current dog study, Flt3-L was administered pretransplant and stopped on day 5 after transplantation, which may have limited the GVH reaction. Another reason why the GVHD may have been less severe in dogs is that host T cells may resist GVHD (Blazar, B., et al., J Immunol 2000; 165:4901). The incidence of severe GVHD in the canine mixed chimeras has been less than 10%. Although Flt3-L may increase GVH alloreactivity, the development of severe GVHD after allogeneic HSCT may depend on other important factors.

Mixed hematopoietic chimerism is a tolerant immunologic state in the GVH and the HVG directions. In preclinical studies of both solid-organ and marrow transplantation, the establishment of mixed chimerism has been associated with the development of tolerance to solid-organ grafts (Schwarze, M., et al., Ann Thorac Surg 2000; 70:131; Kuhr, C., et al., Transplantation 2002; 73:1487). In these established canine mixed chimeras, a state of tolerance was established in the hematopoietic system that resulted in specific tolerance to skin grafts from the marrow donor.

The studies presented herein show that Flt3-L promoted engraftment after transplantation, and the proposed mechanism for this effect is an augmentation of the GVH reaction (without being bound by theory). In this canine model of HSCT, Flt3-L was not associated with the development of severe GVHD.

1. A method, comprising:
(a) administering a Flt3-L composition to a patient in need of a hematopoietic cell transplant;
(b) administering a nonmyeloablative conditioning regimen to the patient; and
(c) transplanting hematopoietic cells to the patient.

2. The method of claim 1, wherein the step of administering a Flt3-L composition is selected from the group consisting of:
(a) administering the Flt3-L composition prior to transplanting hematopoietic cells to the patient;
(b) administering the Flt3-L composition concurrent with transplanting hematopoietic cells to the patient;
(c) administering the Flt3-L composition subsequent to transplanting hematopoietic cells to the patient; and
(d) any combination of (a)-(c).

3. The method of claim 1, further comprising administering a growth factor or cytokine.

4. The method of claim 1, wherein the step of administering a nonmyeloablative conditioning regimen is selected from the group consisting of:
(a) radiotherapy;
(b) chemotherapy;
(c) immunosuppression; and
(d) a combination of (a)-(c).

5. The method of claim 1, further comprising administering one or more immunosuppressants to the patient post-transplant.

6. The method of claim 1, further comprising administering adoptive immunotherapy post-transplant.

7. The method of claim 6, wherein the adoptive immunotherapy is a donor lymphocyte infusion.

8. A method, comprising:
(a) administering a Flt3-L composition to a patient in need of a hematopoietic cell transplant,
(b) administering nonmyeloablative conditioning regimen to the patient; and
(c) transplanting hematopoietic cells to the patient, wherein the hematopoietic cells are autologous hematopoietic cells.

9. The method of claim 8, wherein the step of administering a Flt3-L composition is selected from the group consisting of:
(a) administering the Flt3-L composition prior to transplanting hematopoietic cells to the patient;
(b) administering the Flt3-L composition concurrent with transplanting hematopoietic cells to the patient;
(c) administering the Flt3-L composition subsequent to transplanting hematopoietic cells to the patient; and
(d) any combination of (a)-(c).

10. The method of claims 8, further comprising administering a growth factor or cytokine.

11. The method of claims 8, wherein the autologous cells are obtained from autologous bone marrow.

12. The method of claims 8, wherein the autologous cells are obtained from autologous peripheral blood.

13. The method of claims 8, wherein the step of administering a nonmyeloablative conditioning regimen is selected from the group consisting of:
(a) radiotherapy;
(b) chemotherapy;
(c) immunosuppression; and
(d) a combination of (a)-(c).

14. The method of claim 8, further comprising administering one or more immunosuppressants to the patient post-transplant.

15. The method of claim 8, further comprising administering adoptive immunotherapy post-transplant.

16. The method of claim 15, wherein the adoptive immunotherapy is a donor lymphocyte infusion.

17. A method, comprising:
(a) administering a Flt3-L composition to a patient in need of a hematopoietic cell transplant,
(b) administering a nonmyeloablative conditioning regimen to the patient; and
(c) transplanting hematopoietic cells to the patient, wherein the hematopoietic cells are allogeneic hematopoietic cells.

18. The method of claim 17, wherein the step of administering a Flt3-L composition is selected from the group consisting of:
(a) administering the Flt3-L composition prior to transplanting hematopoietic cells to the patient;
(b) administering the Flt3-L composition concurrent with transplanting hematopoietic cells to the patient;
(c) administering the Flt3-L composition subsequent to transplanting hematopoietic cells to the patient; and
(d) any combination of (a)-(c).

19. The method of claim 17, further comprising administering a growth factor or cytokine.

20. The method of claims 17, wherein the allogeneic cells are obtained from allogeneic bone marrow.

21. The method of claims 17, wherein the allogeneic cells are obtained from allogeneic peripheral blood.

22. The method of claim 17, wherein the step of administering the a nonmyeloablative conditioning regimen is selected from the group consisting of:
(a) radiotherapy;
(b) chemotherapy;
(c) immunosuppression; and
(d) a combination of (a)-(c).

23. The method of claim 17, further comprising administering one or more immunosuppressants to the patient post-transplant.

24. The method of claim 17, further comprising administering adoptive immunotherapy post-transplant.

25. The method of claim 24, wherein the adoptive immunotherapy is a donor lymphocyte infusion.


SEQUENCE LISTING (8 sequences)

SEQ ID NO: 1 - DNA, 879 bp, Homo sapien
  CDS (57)..(752)
gtcgactggaacgagacgacctgctctgtcacaggcatgaggggtccccggcagagatgacagtgctggcgccagcctggagcccaaattcctccctgttgctgctgttgctgctgctgagtccttgcctgcgggggacacctgactgttacttcagccacagtcccatctcctccaacttcaaagtgaagtttagagagttgactgaccacctgcttaaagattacccagtcactgtggccgtcaatcttcaggacgagaagcactgcaaggccttgtggagcctcttcctagcccagcgctggatagagcaactgaagactgtggcagggtctaagatgcaaacgcttctggaggacgtcaacaccgagatacattttgtcacctcatgtaccttccagcccctaccagaatgtctgcgattcgtccagaccaacatctcccacctcctgaaggacacctgcacacagctgcttgctctgaagccctgtatcgggaaggcctgccagaatttctctcggtgcctggaggtgcagtgccagccggactcctccaccctgctgcccccaaggagtcccatagccctagaagccacggagctcccagagcctcggcccaggcagctgttgctcctgctgctgctgctgcctctcacactggtgctgctggcagccgcctggggccttcgctggcaaagggcaagaaggaggggggagctccaccctggggtgcccctcccctcccatccctaggattcgagccttgtgcatcgttgactcagccagggtcttatctcggttacacctgtaatctcagcccttgggagcccagagcaggattgctgaatggtctggagcaggtcgtctcgttccagtcgac

SEQ ID NO: 2 - protein, 231 aa, Homo sapien
MTVLAPAWSPNSSLLLLLLLLSPCLRGTPDCYFSHSPISSNFKVKFRELTDHLLKDYPVTVAVNLQDEKHCKALWSLFLAQRWIEQLKTVAGSKMQTLLEDVNTEIHFVTSCTFQPLPECLRFVQTNISHLLKDTCTQLLALKPCIGKACQNFSRCLEVQCQPDSSTLLPPRSPIALEATELPEPRPRQLLLLLLLLPLTLVLLAAAWGLRWQRARRRGELHPGVPLPSHP

SEQ ID NO: 3 - DNA, 24 bp, Homo sapien
tcgactggaacgagacgacctgct

SEQ ID NO: 4 - DNA, 20 bp, Homo sapien
agcaggtcgtctcgttccag

SEQ ID NO: 5 - DNA, 988 bp, Homo sapien
  CDS (30)..(734)
cggccggaattccggggcccccggccgaaatgacagtgctggcgccagcctggagcccaacaacctatctcctcctgctgctgctgctgagctcgggactcagtgggacccaggactgctccttccaacacagccccatctcctccgacttcgctgtcaaaatccgtgagctgtctgactacctgcttcaagattacccagtcaccgtggcctccaacctgcaggacgaggagctctgcgggggcctctggcggctggtcctggcacagcgctggatggagcggctcaagactgtcgctgggtccaagatgcaaggcttgctggagcgcgtgaacacggagatacactttgtcaccaaatgtgcctttcagcccccccccagctgtcttcgcttcgtccagaccaacatctcccgcctcctgcaggagacctccgagcagctggtggcgctgaagccctggatcactcgccagaacttctcccggtgcctggagctgcagtgtcagcccgactcctcaaccctgccacccccatggagtccccggcccctggaggccacagccccgacagccccgcagccccctctgctcctcctactgctgctgcccgtgggcctcctgctgctggccgctgcctggtgcctgcactggcagaggacgcggcggaggacaccccgccctggggagcaggtgccccccgtccccagtccccaggacctgctgcttgtggagcactgacctggccaaggcctcatcctgcggagccttaaacaacgcagtgagacagacatctatcatcccattttacaggggaggatactgaggcacacagaggggagtcaccagccagaggatgtatagcctggacacagaggaagttggctagaggccggtcccttccttgggcccctctcattccctccccagaatggaggcaacgccagaatccagcaccggccccatttacccaactctgaacaaagcccccg

SEQ ID NO: 6 - protein, 235 aa, Homo sapien
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLHWQRTRRRTPRPGEQVPPVPSPQDLLLVEH

SEQ ID NO: 7 - DNA, 71 bp, Homo sapien
aattggtacctttggataaaagagactacaaggacgacgatgacaagacacctgactgttacttcagccac

SEQ ID NO: 8 - DNA, 37 bp, Homo sapien
atatggatccctactgcctgggccgaggctctgggag